(19) United States
(12) Patent Application Publication
(10) Pub. No.: US 2013/0095059 A1
(43) Pub. Date: Apr. 18, 2013

(54) INHALED NO DONOR KMUPS DERIVATIVE PREVENTING ALLERGIC PULMONARY VASCULAR AND BRONCHIAL INFLAMMATION VIA SUPPRESSED CYTOKINES, INOS AND INFLAMMATORY CELL COUNTS IN ASTHMA MODEL

(71) Applicant: Ing-Jun CHEN, Kaohsiung City (TW)

(72) Inventor: Ing-Jun CHEN, Kaohsiung City (TW)

(73) Assignee: KAOHSIUNG MEDICAL UNIVERSITY, Kaohsiung City (TW)

(21) Appl. No.: 13/437,550

(22) Filed: Apr. 2, 2012

Related U.S. Application Data
(63) Continuation-in-part of application No. 13/095,393, filed on Apr. 27, 2011, now abandoned.
Application No. 13/095,393 is a continuation-in-part of application No. 12/572,519, filed on Oct. 2, 2009, now abandoned.
Application No. 12/572,519 is a continuation-in-part of application No. 11/857,483, filed on Sep. 19, 2007, now abandoned.

(30) Foreign Application Priority Data
Jun. 15, 2007 (TW) 096121950

(51) Int. Cl. A61K 31/522; A61K 31/785; A61K 31/78; A61K 31/465; A61K 31/351

(52) U.S. Cl. CPC A61K 31/522; A61K 31/465; A61K 31/351; A61K 31/78; A61K 31/785
USPC 424/78.29; 514/252.16

(57) ABSTRACT

A method for treating a disease is provided. The method includes steps of: providing a subject in need thereof; and administering one selected from a group consisting of KMUPS compound represented by formula I, a pharmaceutically acceptable salts thereof; and a pharmaceutical composition thereof to the subject in a dosage from 1 to 2.5 milligram per kilogram of body weight,

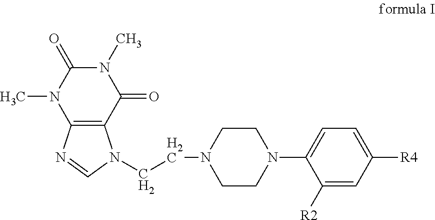

wherein R₂ and R₄ are each selected independently from a group consisting of a C1˜C5 alkoxy group, a hydrogen, a nitro group and a halogen atom.

This application is a continuation-in-part of the application Ser. No. 13/095,393 filed on Apr. 27, 2011, which is a continuation-in-part of application Ser. No. 12/572,519 filed on Oct. 2, 2009, which is a continuation-in-part of application Ser. No. 11/857,483 filed on Sep. 19, 2007, for which priority is claimed under 35 U.S.C. sctn. 120; and this application claims priority of the Application No. 96121950 filed in Taiwan on Jun. 15, 2007 under 35 U.S.C. sctn. 119; the entire contents of all are hereby incorporated by reference.

### FIELD OF THE INVENTION

The present invention relates to a combination therapy method of pharmaceutical composition of a KMUPS derivative and additional active agents capable of preventing allergic pulmonary vascular inflammation and remodeling, and would be useful for the treatment of asthma and respiratory obstruction disease.

### BACKGROUND OF THE INVENTION

Inflammatory processes are prominent in chronic obstructive pulmonary disease (COPD) and hypoxic pulmonary artery hypertension and are increasingly recognized as major pathologic components of pulmonary vascular remodeling. Despite the clinical and functional consequences of peri-bronchial micro-vascular remodeling in asthmatic lungs, up to now, there is little data on therapeutic approaches to this phenomenon.

Anti-inflammatory therapies in the airway are rather ineffective for improving chronic symptoms and reducing inflammation, or reversing the lung function decline and airway remodeling that accompanied a less function in pulmonary vascular system. Specific drug directed against vascular remodeling and chronic inflammation is needed to prevent lung tissue damage and progressive lung function decline

Previously, xanthine-based KMUP-1 has been proved to relax vascular and airway smooth muscle contractions by enhancing endothelial nitric oxide synthase/guanosine 3′,5′-cyclic monophosphate (eNOS/cGMP)-pathway, including increasing soluble guanylyl cyclase α1 (sGCα1) and protein kinase G (PKG) expression (Lin R J, et al., 2006, Wu B N, et al., 2006; Wu B N, et al., 2011; Liu C P, et al., 2012).

Sildenafil has shown anti-inflammatory action against COPD and hypoxic pulmonary artery hypertension, including reducing airway hyper-reactivity, leucocyte influx and elevated NO; its effectiveness was confirmed by measuring elevated S-notroso-N-acetylpenicillamine-induced cGMP generation (Toward T J, et al., 2004, Alp S, et al., 2006). The benefits of sildenafil encouraged us to search for an even more effective eNOS/cGMP-enhancer to inhibit COPD and hypoxic pulmonary artery hypertension.

Endothelial and eNOS/cGMP-signaling is involved in the control of lung function and cell growth. Rapid release of nitric oxide (NO) from the endothelium and epithelium may influence adjacent smooth muscle cell contractility and growth. Endogenous NO produced by activation of eNOS can act as a signaling molecule in several processes, including regulation of pulmonary vascular and airway smooth muscle tone.

Overexpression of eNOS suppresses the features of allergic asthma, originally characterized by expression of inflammatory inducible nitric oxide synthase (iNOS) and exhalation of large NO from airway. In addition, bronchial constriction can cause a pulmonary hypoxic state, reducing vascular endothelial eNOS and decreasing vascular density.

### SUMMARY OF THE INVENTION

In accordance with an aspect of the present invention, a pulmonary inflammatory disease-inhibiting pharmaceutical composition is provided. The pulmonary inflammatory disease-inhibiting pharmaceutical composition includes:

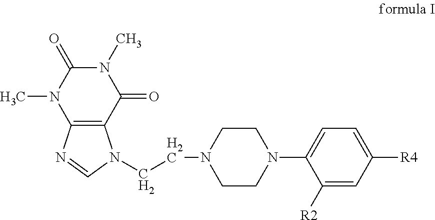

an effective amount of a KMUPS derivative of formula I, wherein R₂ and R₄ are each selected independently from the group consisting of a C1˜C5 alkoxy group, a hydrogen, a nitro group, and a halogen atom; and
a pharmaceutically acceptable carrier.

In accordance with another aspect of the present invention, a method for treating a disease is provided. The method includes steps of:
providing a subject in need thereof; and
administering one selected from a group consisting of KMUPS compound represented by formula I, a pharmaceutically acceptable salts thereof; and a pharmaceutical composition thereof to the subject in a dosage from 1 to 2.5 milligram per kilogram of body weight,

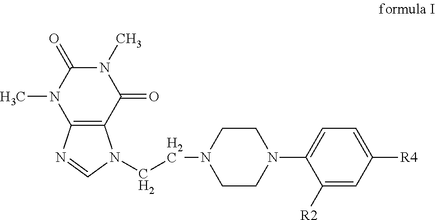

wherein R₂ and R₄ are each selected independently from a group consisting of a C1˜C5 alkoxy group, a hydrogen, a nitro group and a halogen atom.

In accordance with another aspect of the present invention, a method for treating a disease is provided. The method includes a step of administering a therapeutically effective amount of compound being one selected from a group consisting of KMUPS derivative and KMUPS complex compound to a mammal having the disease including one selected from a group consisting of an obstructive pulmonary disease, a pulmonary artery hypertension, pulmonary artery proliferation, an allergic pulmonary vascular inflammation, a vascular remodeling inhibiting disease and a combination thereof.

In accordance with a further aspect of the present invention, a method for treating a disease is provided. The method includes a step of administering a therapeutically effective amount of pharmaceutical composition, in which the active agent is one selected from a group consisting of KMUPS derivative and KMUPS complex compound.

A further aspect for combination is to administrate an effective amount of a KMUPS derivative compound and other administration type of additional active agents to a mammal in need thereof.

A further aspect for combination is to administrate a therapeutically effective amount of pharmaceutical composition, in which the active agent is a KMUPS complex compound, to a mammal in need thereof.

The pulmonary inflammatory disease-inhibiting pharmaceutical composition includes:

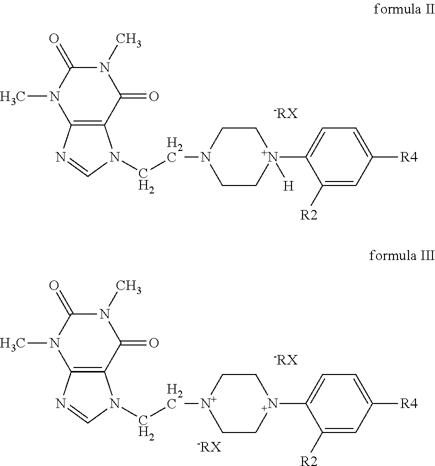

an effective amount of a KMUPS complex compound represented by formula II or formula III, wherein
R₂ and R₄ are each selected independently from the group consisting of a C1˜C5 alkoxy group, a hydrogen, a nitro group, and a halogen atom;
RX includes a carboxylic group which donated from one of a Statin analogues, a Co-polymer, a poly-γ-polyglutamic acid derivative and sodium CMC; and
⁻RX is an anion of a carboxylic group donated from one of a statins analogues, a Co-polymer, a poly-γ-polyglutamic acid derivative and sodium CMC; and
a pharmaceutically acceptable carrier.

KMUP-1 (1.0˜2.5 mM, 30 mins) dose-dependently increases eNOS and decreases MMP-9 expression in mice lung tissues; KMUP-1 shows marked reductions of inflammatory cells, increasing iNOS and MMP-9 and decreasing sGCα1 and PKG expressions; KMUP-1 nebulization significantly reduces the increase in total cells, lymphocytes and eosinophils elicited in the airway lumen 24 hrs after the last ovalbumine challenge; inhaled NO donors KMUPS derivatives prevent allergic pulmonary vascular inflammation; an example of combination treatment or prevention of Allergic Pulmonary Vascular Inflammation.

The above objects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 shows that KMUP-1 decreases plasma 5-HT levels in MCT-PAH;

FIG. 2 shows that KMUP-1 and simvastatin relax 5-HT-induced contraction of pulmonary artery rings;

FIG. 3 shows that Δ[Ca²⁺]i indicates the difference in [Ca²⁺]i between basal and peak levels induced by 5-HT;

FIG. 4 shows that Western blotting revealed a significant difference in expression of 5-HTT after MCT between control and KMUP-1 treatment;

FIG. 5 shows that KMUP-1 inhibited 5-HT-induced 5HTT expression in pulmonary artery smooth muscle cells (PASMCs);

FIG. 6 shows that Y27632 (10 μM) and KMUP-1 (1-100 μM) concentration-dependently inhibited 5-HT-induced expression of 5-HTT;

FIG. 7 shows the comparison of effects of KMUP-1 and simvastatin at 10 μM on 5-HT-induced 5-HTT expression;

FIG. 8 shows the time courses of 5-HT induced RhoA translocation and ROCK expression in PASMCs;

FIG. 9 shows the time courses of 5-HT induced ROCK expression in PASMCs;

FIG. 10 shows that KMUP-1 suppresses 5-HT-induced RhoA translocation;

FIG. 11 shows that KMUP-1 and Y27632 (10 μM) inhibited 5-HT-induced ROCK expression;

FIG. 12 shows that the time courses of 5-HT-stimulated ERK by 5-HT (10 μM) were changed for the indicated time periods in PASMCs;

FIG. 13 shows that the time courses of AKT phosphorylation by 5-HT (10 μM) were changed for the indicated time periods in PASMCs;

FIG. 14 shows that KMUP-1 inhibits 5-HT-induced phosphorylation of ERK1/2;

FIG. 15 shows that KMUP-1 inhibits 5-HT-induced phosphorylation of AKT;

FIG. 16 shows that KMUP-1 inhibited 5-HT-induced migration of PASMCs;

FIG. 17 shows that the cell proliferation was determined by MTT assay;

FIG. 18 shows the effects of KMUP-1 on 5-HT_{2B} and eNOS protein expression in HPAEC;

FIG. 19 shows that KMUP-1 increased the expression of eNOS in 5-HT-treated HPAEC;

FIG. 20 shows that KMUP-1 increased the production of NO in 5-HT-treated HPAEC;

FIG. 21 shows the experimental protocol of short term nebulization;

FIGS. 22A-22B show the effects of short-term inhalation of KMUP-1 and L-NAME-pretreatment on eNOS expression in lung tissues;

FIG. 22A shows that expression of eNOS;

FIG. 22B shows that L-NAME-pretreatment reverses eNOS expression;

FIGS. 23A-23B show the effects of short-term inhalation of KMUP-1 and L-NAME-pretreatment on MMP-9 expression in lung tissues;

FIG. 23A shows that effect of KMUP-1 on MMP-9 expression; FIG. 23B shows that effect of L-NAME-pretreatment on MMP-9 expression;

FIGS. 24A-24F show the morphology of KMUP-1 on ovalbumine-sensitized perivascular and peribronchial lung tissue;

FIG. 24A shows that morphology of control group on peri-vascular;

FIG. 24B shows that morphology of OVA on peri-vascular;

FIG. 24C shows that morphology of OVA+KMUP-1 on peri-vascular;

FIG. 24D shows that morphology of control group on peri-bronchiolar;

FIG. 24E shows that morphology of OVA on peri-bronchiolar;

FIG. 24F shows that morphology of OVA+KMUP-1 on peri-bronchiolar;

FIGS. 25A-25B show the effects of KMUP-1 on ovalbumine-challenged perivascular and peribronchial lung tissue inflammation;

FIG. 25A shows that effects of perivascular and peribronchial lung tissue inflammation;

FIG. 25B shows that effect of peribronchial lung tissue inflammation;

FIGS. 26A-26B show the effects of KMUP-1 on the eNOS and iNOS expression;

FIG. 26A shows that effect of KMUP-1 on the eNOS expression;

FIG. 26B shows that effect of KMUP-1 on the iNOS expression;

FIGS. 27A-27B show the effects of KMUP-1 on the sGCα1 and PKG expression;

FIG. 27A shows that effect of KMUP-1 on the sGCα1 expression;

FIG. 27B shows that effect of KMUP-1 on the PKG expression;

FIGS. 28A-28B show the effects of KMUP-1 on the MMP-9 expression;

FIG. 28A shows that effect of KMUP-1 on the MMP-9 expression;

FIG. 28B shows that effect of KMUP-1 on the Zymography of MMP-9 expression;

FIGS. 29A-29B show the effects of KMUP-1 on the ICAM-1 and VCAM-1 expression;

FIG. 29A shows that effect of KMUP-1 on the ICAM-1 expression;

FIG. 29B shows that effect of KMUP-1 on the VCAM-1 expression;

FIGS. 30(A)-30(H) show the microphotographic demonstration of KMUP-1's effects on OVA-induced eNOS-immunostaining and MMP-9-immunostaining in lung sections;

FIG. 30A shows that morphology of control group;

FIG. 30B shows that morphology of OVA;

FIG. 30C shows that morphology of OVA+KMUP-1;

FIG. 30D shows that morphology of OVA+KMUP-1;

FIG. 30E shows that morphology of OVA+KMUP-1;

FIG. 30F shows that morphology of control group;

FIG. 30G shows that morphology of OVA+KMUP-1;

FIG. 30H shows that morphology of OVA+KMUP-1;

FIGS. 31A-31B shows the effects of KMUP-1 on NOx levels and cellular components in BALF;

FIG. 31A shows that effect of KMUP-1 on NOx levels in BALF;

FIG. 31B shows that effect of KMUP-1 on cellular components in BALF;

FIGS. 32A-32B show the effects of KMUP-1 on OVA-sensitized and challenged mice plasma cytokines;

FIG. 32A shows the effect of KMUP-1 of IL-5 levels; and

FIG. 32B shows the effect of KMUP-1 of IL-13 levels;

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

In an embodiment of the present invention, a combination therapy is disclosed for treating and/or preventing pulmonary disease. The compositions are formulated and administered in the same general manner as detailed herein. The KMUPS derivative compounds represented by structural of formula I may be used effectively alone or in combination with one or more active agents depending on the desired target therapy. Combination therapy includes administration of a single pharmaceutical dosage composition which contains a compound of structural formula I and one or more additional active agents, as well as other administration type of additional active agents, and each active agent in its own separate pharmaceutical dosage formulation.

When used in combination, in some embodiments, the compound of structural formula I may be administered as a single pharmaceutical dosage composition that contains additional active agents. In other embodiments, separate dosage composition are administered; the formula I compound and the other additional pharmaceutical agent may be, administered at essentially the same time, for example, concurrently, or at separately staggered times, for example, sequentially. In certain examples, the individual components of the combination may be administered separately, at different times during the course of therapy, or concurrently, in divided or single combination forms. Also provided is, for example, simultaneous, staggered, or alternating treatment.

For example, a compound of structural formula I can be administered to the patient together in a single oral dosage composition such as a tablet or capsule, or each agent administered in separate oral dosage formulations. Where separate dosage formulations of additional active agents are used, can be administered other administration type at essentially the same time. An example of combination treatment may be by any suitable administration route including oral (including buccal and sublingual), rectal, nasal, airway inhalation (e.g., dry powder or aerosolized formulation), vaginal, and parenteral (including subcutaneous, intramuscular, intravenous and intradermal), topical administration include, but are not limited to, sprays, mists, aerosols, solutions, lotions, gels, creams, ointments, pastes, unguents, emulsion and suspensions, with oral or parenteral being preferred. The preferred route may vary with the condition and age of the recipient.

While it is possible for the administered ingredients to be administered alone, it is preferable to present them as part of a pharmaceutical formulation. The formulations of the present invention comprise the administered ingredients, as defined above, together with one or more acceptable carriers thereof and optionally other additional therapeutic ingredients. The carrier(s) must be “acceptable” in the sense of being compatible with the other ingredients of the formulation and not deleterious to the recipient.

According to the above-mentioned aspect of the present invention, additional active agents may include additional compounds according to the invention, or one or more other pharmaceutically active agents. In preferable embodiments, the inventive compositions will contain the active agents, including the inventive combination of therapeutic agents, in an amount effective to treat an indication of interest.

Preferably, in one embodiment, additional active agents consists of Statin analogues, a Co-polymer, a poly-γ-polyglutamic acid derivative and sodium carboxyl methylcellulose (sodium CMC).

The substance being distributed is suitably administered in any way in which at least some (preferably about 1 wt. % to about 100 wt. %) of the substance reaches the bloodstream of the organism. Thus, the substance can be administered enterally (via the alimentary canal) or parenterally (via any route other than the alimentary canal, such as, e.g., through intravenous injection, subcutaneous injection, intramuscular injection, inhalation percutaneous application, etc.).

According to a further feature of this aspect of the invention there is provided a method for producing a treating effect of pulmonary disease in a warm-blooded animal, such as man, in need of such treatment which comprises administering to said animal an effective amount of a KMUPS derivative compounds or a KMUPS complex compound, or a pharmaceutical acceptable salt, solvate, solvate of such a salt or a prodrug thereof.

A pharmaceutical composition is provided in the present, in which the active agent is a theophylline-based moiety compound for treating a pulmonary disease, and KMUPS derivative compound has the activity of NO includes cGMP-dependent and cGMP-independent.

A theophylline-based moiety compound, i.e. KMUPS derivative, which is obtained by reacting theophylline compound with piperazine compound and then recrystallizing the intermediate therefrom, is provided in the present invention. KMUPS derivative compound has the activities for treating a pulmonary disease and the benefits of good solubility, low toxicity and safety.

Preferably, The pharmaceutical composition includes one of a KMUPS derivative compound having a formula I or its salts,

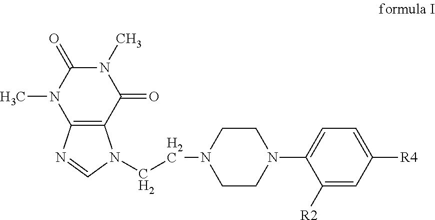

wherein R₂ and R₄ are each selected independently from the group consisting of a C1˜C5 alkoxy group, a hydrogen, a nitro group, and a halogen atom. The above-mentioned halogen refers to fluorine, chlorine, bromine and iodine.

Preferably, in one embodiment, the compound of formula I is KMUP-1, wherein R₂ is chloro atom and R₄ is hydrogen, which has the generally chemical name 7-[2-[4-(2-chlorophenyl)piperazinyl]ethyl]-1,3-dimethylxanthine. The compound of formula I is KMUP-2, wherein R₂ is methoxy group and R₄ is hydrogen, which has the chemical name 7-[2-[4-(2-methoxybenzene)piperazinyl]ethyl]-1,3-dimethylxanthine. In another embodiment, the compound of formula I also is KMUP-3, wherein R₂ is hydrogen and R₄ is nitro group, which has the chemical name 7-[2-[4-(4-nitrobenzene)piperazinyl]ethyl]-1,3-dimethylxanthine. In another embodiment, the compound of formula I also is KMUP-4, wherein R₂ is nitro group and R₄ is hydrogen, which has the chemical name 7-[2-[4-(2-nitrobenzene)piperazinyl]ethyl]-1,3-dimethylxanthine.

The present invention provides a KMUPS complex compound having a formula II or formula III,

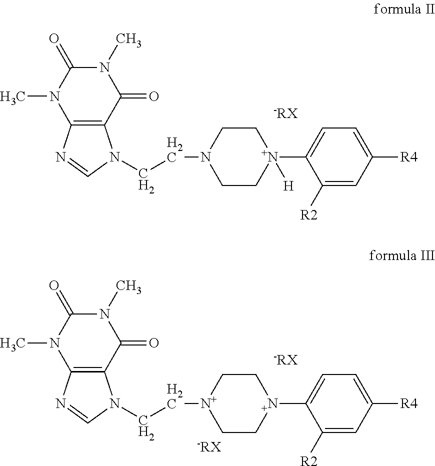

wherein R₂ and R₄ are each selected independently from the group consisting of a C1˜C5 alkoxy group, a hydrogen, a nitro group, and a halogen atom;
RX includes a carboxylic group which donated from one of a Statin analogues, a Co-polymer, a poly-γ-polyglutamic acid derivative and sodium CMC; and
⁻RX is an anion of a carboxylic group donated from one of a statins analogues, a Co-polymer, a poly-γ-polyglutamic acid derivative and sodium CMC.

Preferably, the above statins analogues may be commercial available statin derivative drugs, including Atorvastatin, Cerivastatin, Fluvastatin, Lovastatin, Mevastatin, Pravastatin, Rosuvastatin, Simvastatin and Pramastatinic acid. Co-polymers is one selected from a group consisting of a hyaluronic acid, a polyacrylic acid, a polymethacrylates (PMMA), an Eudragit, a dextran sulfate, a heparan sulfate, a polylactic acid or polylactide (PLA), a polylactic acid sodium (PLA sodium) and a polyglycolic acid sodium (PGA sodium). Poly-γ-polyglutamic acid (γ-PGA) derivative is one selected from a group consisting of an alginate sodium, a poly-γ-polyglutamic acid sodium (γ-PGA sodium), a poly-γ-polyglutamic acid (γ-PGA) and an alginate-poly-lysine-alginate (APA). Hyaluronic Acid is a polymer, which are composed of alternating units of N-acetyl glucosamine (NAG) and D-glucuronic acid. Eudragit is a trade name of series copolymers derived from esters of acrylic and methacrylate acid.

The term “KMUPS derivative compound” as used herein refers to one selected from a group consisting of KMUP-1, KMUP-2, KMUP-3, KMUP-4 and its pharmaceutical acceptable salts. Preferably, the pharmaceutical composition further includes at least one of a pharmaceutically acceptable carrier and an excipient.

To achieve the above purpose, formula I salts, formula II and formula III can synthetically produced from the 2-Chloroethyltheophylline compound and piperazine substituted compound.

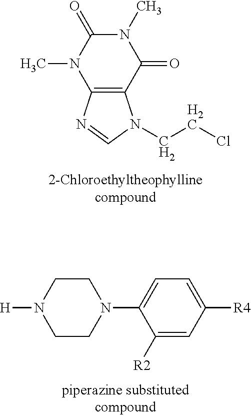

The compounds of formula I salts and KMUPS complex compound set forth in the examples below were prepared using the following general procedures as indicated.

The general procedure 1 includes steps of dissolving 2-Chloroethyl theophylline and piperazine substituted compound in hydrous ethanol solution, and the amount of reagent should be conjugated depending on the molecular weight percentage. After adding the strong base e.g. sodium hydroxide (NaOH) or sodium hydrogen carbonate (NaHCO₃) to make the solution more alkaline or more basic, a heating procedure is performed under reflux for three hours. Allowed to stand overnight, the cold supernatant was decanted for proceeding, efficient removal of solvents by vacuum concentration, and then the residue were dissolved with one-fold volume of ethanol and three-fold volume of 2N hydrochloric acid (HCl), kept at 50° C. to 60° C. to make a saturated solution (pH 1.2). The saturated solution was sequentially treated, decolorized with activated charcoal, filtered, deposited overnight and filtered to obtain KMUP-1 HCl with a white crystal.

The general procedure 2 includes steps of dissolving 2-Chloroethyl theophylline and piperazine substituted compound in hydrous ethanol solution, and the amount of reagent should be conjugated depending on the molecular weight percentage. Then, a heating procedure is performed under reflux for three hours. Allowed to stand overnight, the cold supernatant was decanted for proceeding, efficient removal of solvents by vacuum concentration, and then the residue were dissolved with one-fold volume of ethanol and three-fold volume of 2N hydrochloric acid (HCl), kept at 50° C. to 60° C. to make a saturated solution (pH 1.2). The saturated solution was sequentially treated, decolorized with activated charcoal, filtered, deposited overnight and filtered to obtain KMUP-1 HCl with a white crystal.

According to the general procedure 1 or 2, KMUPS derivative of formula I can be synthetically produced directly, from the 2-Chloroethyltheophylline compound and piperazine substituted compound. Preferably, a theophylline-based moiety compound derivative, i.e. KMUPS derivative, which is obtained by reacting theophylline compound with piperazine compound and then recrystallizing the intermediate therefrom, is provided in the present invention.

Preferably, the pharmaceutical acceptable salts of KMUP-1, KMUP-2, KMUP-3 and KMUP-4 are citric acid, nicotinic acid and hydrochloride.

Thereby, KMUPS compound may represent KMUPS complex compound. Preferably, in one embodiment, KMUP-1 is dissolved in a mixture of ethanol and γ-Polyglutamic acid. The solution is reacted at warmer temperature, the methanol is added thereinto under room temperature, and the solution is incubated over night for crystallization and filtrated to obtain KMUP-1-γ-Polyglutamic complex. According to the above-mentioned general procedure 1 or 2, a sufficient amount of KMUP derivative may react with containing a carboxyl group of “RX” to form a formula II of KMUPS complex compound. Further, the term “RX” group can be referred to the Statin analogues, Co-polymer, poly-γ-polyglutamic acid derivative or sodium CMC, which contains sufficient amount of the carboxyl group and can react with the piperazine group of KMUP derivative to prepare the above formula III of KMUPS complex according to the method in the present invention. The synthesized KMUPS complex compound may show a pro-drug and multiple therapeutic functions in the body via a chemical or an enzymatic hydrolysis.

Specifically speaking, KMUPs complex compounds, in one embodiment, as KMUP-1-Atorvastatin complex, KMUP-2-Atorvastatin complex, KMUP-3-Atorvastatin complex, KMUP-4-Atorvastatin complex; KMUP-1-Cerivastatin complex, KMUP-2-Cerivastatin complex, KMUP-3-Cerivastatin complex, KMUP-4-Cerivastatin complex; KMUP-1-Fluvastatin complex, KMUP-2-Fluvastatin complex, KMUP-3-Fluvastatin complex, KMUP-4-Fluvastatin complex; KMUP-1-Lovastatin complex, KMUP-2-Lovastatin complex, KMUP-3-Lovastatin complex, KMUP-4-Lovastatin complex; KMUP-1-Mevastatin complex, KMUP-2-Mevastatin complex, KMUP-3-Mevastatin complex, KMUP-4-Mevastatin complex; KMUP-1-Pravastatin complex, KMUP-2-Pravastatin complex, KMUP-3-Pravastatin complex, KMUP-4-Pravastatin complex; KMUP-1-Rosuvastatin complex, KMUP-2-Rosuvastatin complex, KMUP-3-Rosuvastatin complex, KMUP-4-Rosuvastatin complex; KMUP-1-Simvastatin complex, KMUP-2-Simvastatin complex, KMUP-3-Simvastatin complex, KMUP-4-Simvastatin complex; KMUP-1-CMC complex, KMUP-2-CMC complex, KMUP-3-CMC complex, KMUP-4-CMC complex; KMUP-1-hyaluronic complex, KMUP-2-hyaluronic complex, KMUP-3-hyaluronic complex, KMUP-4-hyaluronic complex; KMUP-1-polyacrylic complex, KMUP-2-polyacrylic complex, KMUP-3-polyacrylic complex, KMUP-4-polyacrylic complex; KMUP-1-Eudragit complex, KMUP-2-Eudragit complex, KMUP-3-Eudragit complex, KMUP-4-Eudragit complex; KMUP-1-polylactide complex, KMUP-2-polylactide complex, KMUP-3-polylactide complex, KMUP-4-polylactide complex; KMUP-1-polyglycolic complex, KMUP-2-polyglycolic complex, KMUP-3-polyglycolic complex, KMUP-4-polyglycolic complex; KMUP-1-dextran sulfate complex, KMUP-2-dextran sulfate complex, KMUP-3-dextran sulfate complex, KMUP-4-dextran sulfate complex; KMUP-1-heparan sulfate complex, KMUP-2-heparan sulfate complex, KMUP-3-heparan sulfate complex, KMUP-4-heparan sulfate complex; KMUP-1-alginate complex, KMUP-2-alginate complex, KMUP-3-alginate complex, KMUP-4-alginate complex; KMUP-1-γ-PGA complex, KMUP-2-γ-PGA complex, KMUP-3-γ-PGA complex, KMUP-4-γ-PGA complex; KMUP-1-APA complex, KMUP-2-APA complex, KMUP-3-APA complex, KMUP-4-APA complex etc.

In accordance with a further aspect of the present invention, depending on the desired clinical use and the effect, the adaptable administration method of KMUPS derivative and KMUPS complex pharmaceutical composition includes one selected from a group consisting of an oral administration, an intravenous injection, a subcutaneous injection, an intraperitoneal injection, an intramuscular injection and a sublingual administration.

Preferably, in one embodiment, KMUPS derivative and KMUPS complex compounding aerosol formulations also attempt to use for administration. The use of aerosols to administer medicaments has been known for several decades. Such aerosols generally comprise the medicament, one or more propellants and either a surfactant or a solvent, such as ethanol. The majority of available pressurized metered-dose inhaler (pMDI) devices contain chlorofluorocarbon (CFC) propellants, which have well-documented adverse effects on the atmospheric ozone layer. Over the past few years, research has led to the development and approval of hydrofluoroalkane (HFA)-based aerosols, which do not have ozone-depleting properties, as alternatives to CFC propellants.

Thus, for example, EP 0372777 requires the use of 1,1,1,2-tetrafluoroethane in combination with both a cosolvent having greater polarity than 1,1,1,2-tetrafluoroethane (e.g. an alcohol or a lower alkane) and a surfactant in order to achieve a stable formulation of a medicament powder. In particular, it is noted from the specification on Page 3, Line 7 that “it has been found that the use of propellant 134a (1,1,1,2-tetrafluoroethane) and drug as a binary mixture or in combination with a conventional surfactant such as sorbitan trioleate does not provide formulation having suitable properties for use with pressurised inhalers”. Surfactants are generally recognized by those skilled in the art to be essential components of aerosol formulation, required not only to reduce aggregation of the medicament but also to lubricate the valve employed, thereby ensuring consistent reproducibility of valve actuation and accuracy of dose dispensed. Whilst WO91/11173, WO91/11495 and WO91/14422 are concerned with formulation comprising an admixture of drug and surfactant. WO91/04011 discloses medicinal aerosol formulation in which the particulate medicaments are pre-coated with surfactant prior to dispersal in 1,1,1,2-tetrafluoroethane.

We have now surprisingly found that, in contradistinction to these teachings, it is in fact possible to obtain satisfactory dispersions of medicaments in fluorocarbon or hydrogen-containing chlorofluorocarbon propellants such as 1,1,1,2-tetrafluoroethane without recourse to the use of any surfactant or cosolvent in the composition, or the necessity to pre-treat the medicament prior to dispersal in the propellant.

The term excipients or “pharmaceutically acceptable carrier or excipients” and “bio-available carriers or excipients” mentioned above include any appropriate compounds known to be used for preparing the dosage form, such as the solvent, the dispersing agent, the coating, the anti-bacterial or anti-fungal agent and the preserving agent or the delayed absorbent. Usually, such kind of carrier or excipient does not have the therapeutic activity itself. Each formulation prepared by combining the derivatives disclosed in the present invention and the pharmaceutically acceptable carriers or excipients will not cause the undesired effect, allergy or other inappropriate effects while being administered to an animal or human. Accordingly, the derivatives disclosed in the present invention in combination with the pharmaceutically acceptable carrier or excipients are adaptable in the clinical usage and in the human.

A therapeutic effect can be achieved by using the suitable dosage forms, in part, depend upon the use or the route of administration, eg. venous, oral, and inhalation routes or via the nasal, rectal and vaginal routes. Such dosage forms should allow the compounds to reach target cells. About 0.1 mg to 1000 mg per day of the active ingredient is administered for the patients of various diseases. Preferably, in one embodiment, single oral dose of KMUPS derivative or KMUPS complex compound is about 1˜2.5 milligram per kilogram of body weight. In the treatment of respiratory conditions, the KMUPS derivative compound and KMUPS complex compound are preferably delivered by inhalation. The compound can be delivered as an aerosol, mist or powder. An effective amount for delivery by inhalation is about 2.5˜5 mM per inhalation, several times daily. The compound also can be delivered orally in amounts of about 1˜2.5 milligram per kilogram of body weight. The use of single dose oral combination therapy, the amounts of KMUPS derivative and additional active agents in a dosage form may be administered amounts of about 10˜50 milligram as a single pharmaceutical dosage composition.

The carrier is varied with each formulation, and the sterile injection composition can be dissolved or suspended in the non-toxic intravenous injection diluents or solvent such as 1,3-butanediol. Among these carriers, the acceptable carrier may be mannitol or water. Besides, the fixing oil or the synthetic glycerol ester or di-glycerol ester is the commonly used solvent. The fatty acid such as the oleic acid, the olive oil or the castor oil and the glycerol ester derivatives thereof, especially the oxy-acetylated type, may serve as the oil for preparing the injection and as the naturally pharmaceutical acceptable oil. Such oil solution or suspension may include the long chain alcohol diluents or the dispersing agent, the carboxylmethyl cellulose or the analogous dispersing agent. Other carriers are common surfactant such as Tween and Spans or other analogous emulsion, or the pharmaceutically acceptable solid, liquid or other bio-available enhancing agent used for developing the formulation that is used in the pharmaceutical industry.

The composition for oral administration adopts any oral acceptable formulation, which includes capsule, tablet, pill, emulsion, aqueous suspension, dispersing agent and solvent. The carrier is generally used in the oral formulation. Taking the tablet as an example, the carrier may be the lactose, the corn starch and the lubricant, and the magnesium stearate is the basic additive. The diluents used in the capsule include the lactose and the dried corn starch. For preparing the aqueous suspension or the emulsion formulation, the active ingredient is suspended or dissolved in an oil interface in combination with the emulsion or the suspending agent, and the appropriate amount of the sweetening agent, the flavors or the pigment is added as needed.

The nasal aerosol or inhalation composition may be prepared according to the well-known preparation techniques. For example, the bioavailability can be increased by dissolving the composition in the phosphate buffer saline and adding the benzyl alcohol or other appropriate preservative, or the absorption enhancing agent. The compound of the present invention may be formulated as suppositories for rectal or virginal administration.

The compound of the present invention can also be administered intravenously, as well as subcutaneously, parentally, muscular, or by the intra-articular, intracranial, intra-articular fluid and intra-spinal injections, the aortic injection, the sterna injection, the intra-lesion injection or other appropriate administrations.

The present invention provides a pharmaceutical composition in which the active agent is a theophylline-based moiety compound for inhibiting monocrotaline (MCT)-induced pulmonary artery proliferation by binding to 5-HT_{2A}, 5-HT_{2B} and 5-HT_{2C} receptors, increasing endothelial eNOS/5-HT_{2B} receptor expression and NO release and inhibiting 5-HTT/RhoA/ROCK expression and AKT/ERK phosphorylation. KMUPS is suggested to be useful in the treatment of 5-HT-induced pulmonary artery proliferation. In particular, they are related to obstructive pulmonary disease, pulmonary artery hypertension, pulmonary artery proliferation and vascular remodeling inhibiting disease.

To achieve the above purpose, the Ca²⁺ sensitivity of PASMCs is extremely important for pulmonary artery contraction. MCT-released 5-HT can increase the [Ca²⁺]i of PASMCs to enhance the vasoconstrictor response. The increase of [Ca²⁺]i promotes the proliferation of PASMC via immediate early genes. In most cases, cell proliferation requires external calcium and is inhibited when cells are cultured in a Ca²⁺-deprived or Ca²⁺ channel blocker-supplemented medium. The role of ion channels in PASMCs proliferation is currently under investigation, since the link between proliferation and ion channel activation could be a therapeutic target in clinical pulmonary artery hypertension (PAH). In PASMCs, 5-HT exerts vasoconstrictor and mitogenic effects, both dependent on an increase of cytosolic [Ca²⁺]i. 5-HT increases [Ca²⁺]i in PASMCs and causes pulmonary artery contraction, which is reduced by KMUP-1. pulmonary artery hypertension (PAH) is characterized by high circulating 5-HT levels, 5-HT-induced hyper-reactivity and SMC proliferation, suggesting a major role for 5-HT in both vascular wall remodeling and elevated vascular resistance. Sustained Ca²⁺ elevation contributes to both PASMC contraction and proliferation. Increases of [Ca²⁺]i have been demonstrated to cause excessive proliferation of PASMCs in PAH, while abnormal intracellular Ca²⁺ sequestration is thought to be linked to smooth muscle dysfunction in patients with chronic obstructive pulmonary disease. We therefore suggest that the pulmonary artery relaxation and anti-proliferation effects of KMUPS include the inhibition of Ca²⁺-influx.

For proving the treating effect of pulmonary disease the KMUPS pharmaceutically acceptable salt, using both the chronic model of PAH induced by monocrotaline (MCT) in rats. So as to confirm that KMUPS treatment would inhibit PAH, via cGMP-dependent inhibition of RhoA/ROCK II (Rho kinase II) in pulmonary artery and lung tissue.

Chronic PAH

Rats treated with vehicle once a day for 21 days after a single intra-peritoneal injection of MCT developed PAH. Long-term daily treatment with KMUP-1 (5 mg/kg/day p.o. and 1 mg/kg/day i.p.) for 21 days significantly reduced MCT-induced increases in mean pulmonary arterial pressure (MPAP) as shown previously.

Plasma 5-HT Levels in MCT-Treated Rats

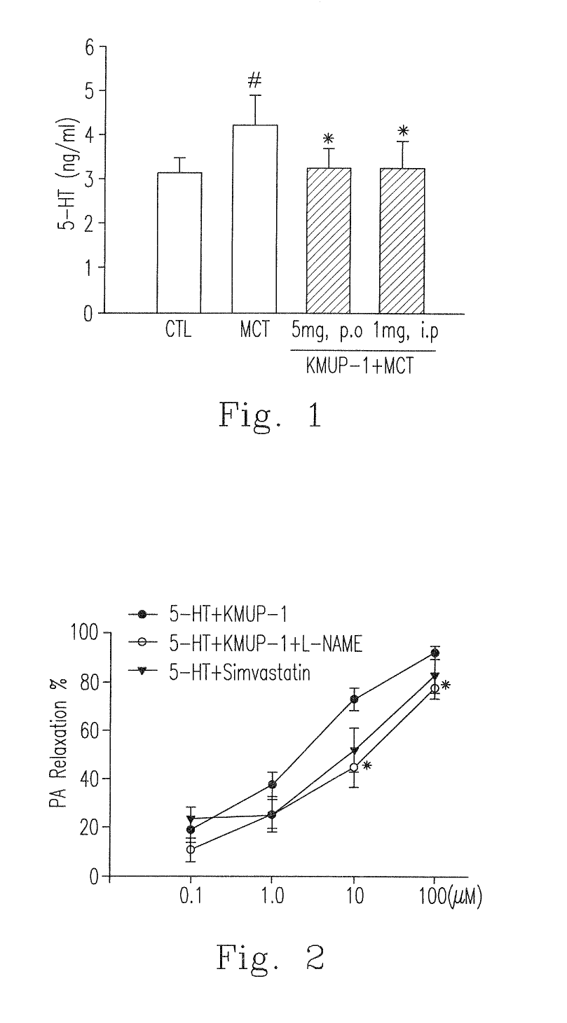

The plasma concentrations of 5-HT are shown in FIG. 1. Administration of KMUP-1 (5 mg/kg p.o., 1 mg/kg i.p.) prevents increases in plasma 5-HT levels. Data are representative of 6 experiments. The plasma concentration of 5-HT was significantly greater in MCT-PAH rats than control rats (4.2±0.7 and 3.2±0.3 ng/mL, respectively; P<0.05). The plasma concentration of 5-HT in KMUP-1-treated rats was significantly decreased, compared to non-treated MCT-PAH rats (3.2±0.5 ng/mL, p.o. and 3.2±0.6 ng/mL, i.p. 1 mg/kg, respectively; P<0.05) (FIG. 1).

Effects on 5-HT-Constricted Pulmonary Artery

5-HT (10 μM) produced a contractile response in pulmonary artery of control group, which was inhibited by KMUP-1 (0.1-100 μM) and simvastatin (0.1-100 μM). In the presence of the NOS inhibitor L-NAME (100 μM), KMUP-1 (10-100 μM) reduced the maximum response of pulmonary artery to 5-HT (P<0.05). L-NAME itself did not induce any constriction, but it enhanced 5-HT-induced constriction. KMUP-1 reversed the 5-HT-induced constriction in rat pulmonary artery in a concentration-dependent manner. L-NAME did not significantly reduce this reversal (FIG. 2).

Intracellular Calcium Response to 5-HT

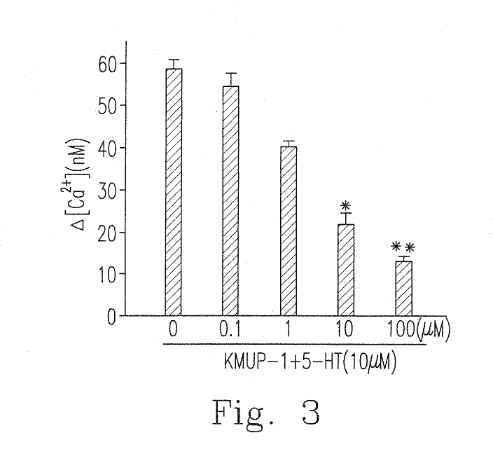

5-HT (10 μM) induced a calcium influx in PASMCs. In the following set of experiments, KMUP-1 inhibited Ca²⁺-influx induced by 5-HT in PASMCs. 5-HT (10 μM) caused a significantly release of [Ca²⁺]i. KMUP-1 (0.1-100 μM) concentration-dependently inhibited elevation of [Ca²⁺]i. Δ[Ca²⁺]i indicates the difference in [Ca²⁺]i between basal and peak treated levels induced by 5-HT (FIG. 3). KMUP-1 significantly inhibited the sustained [Ca²⁺]i response to 5-HT by 65% at 10 μM.

Effect of KMUP-1 on PASMC Proliferation in MCT-Treated Rats

Rat lung PASMC proliferation (PCNA) on day 21 after MCT injection. Medial hypertrophy was associated with an increased number of proliferating vascular cells, shown by immunohistochemistry for PCNA. In MCT-treated rats administered with vehicle, PCNA labeling indicated the proliferation of PASMCs in distal pulmonary artery walls, which were more marked in MCT-treated groups. The number of PCNA-positive cells was markedly lower in the pulmonary artery walls of rats treated with KMUP-1.

Pulmonary 5-HTT expression and vascular immunochemistry

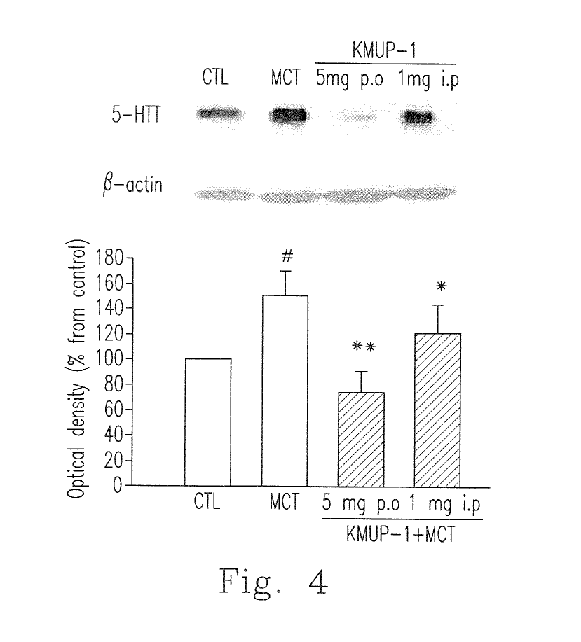

5-HTT expression in pulmonary artery 21 days after MCT injection was significantly decreased after treatment with KMUP-1 at doses of 5 mg/kg p.o. and 1 mg/kg i.p., as assayed by immunochemistry. Western blotting measurement of 5-HTT protein expression in lung showed similar results. 5-HTT protein expression was significantly reduced by administration of KMUP-1 in lung tissue (FIG. 4).

5-HTT Expression in PASMCs

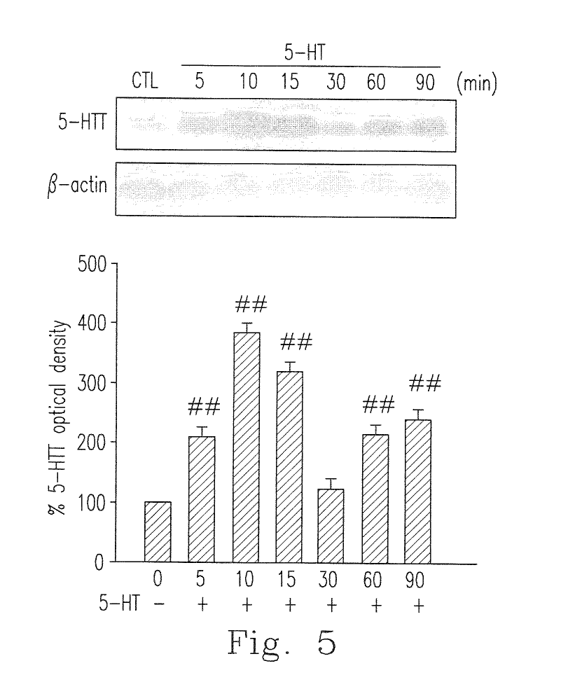

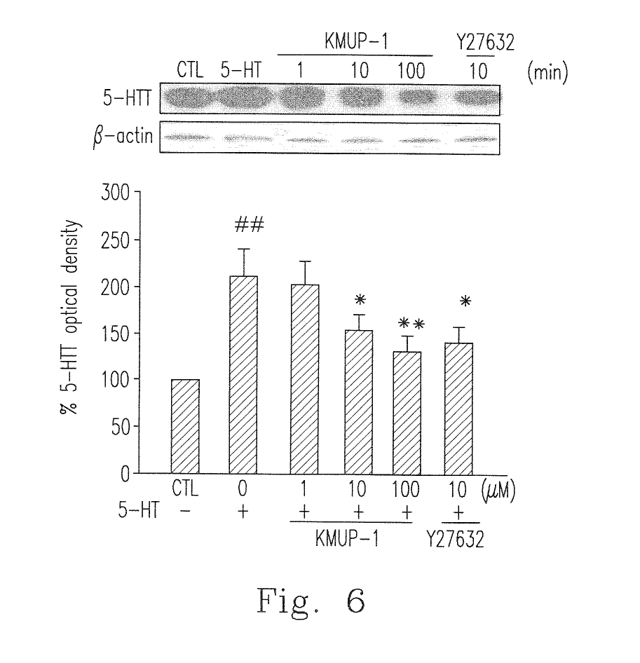

PASMCs were treated with 5-HT (10 μM) for 5-90 min. We found that 5-HTT protein expression achieved a peak 10 min after 5-HT treatment (FIG. 5). Cells were first treated with KMUP-1 (1-100) 1M) and Rho kinase (ROCK) inhibitor Y27632 (10 μM) for 24 h and then with 5-HT for 10 min. Y27632 (10 μM) and KMUP-1 dose-dependently inhibited 5-HT-induced expression of 5-HTT (FIG. 6). KMUP-1 and simvastatin at 10 μM also both inhibited 5-HT-induced 5-HTT expression (FIG. 7).

RhoA Translocation and ROCK Expression in PASMCs

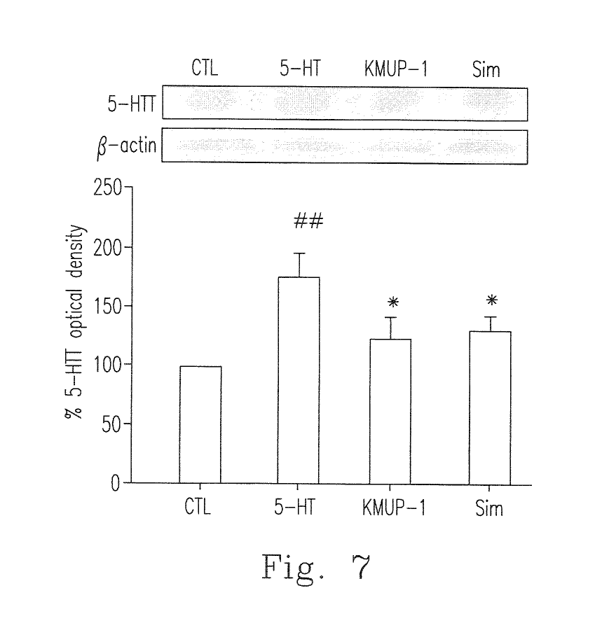

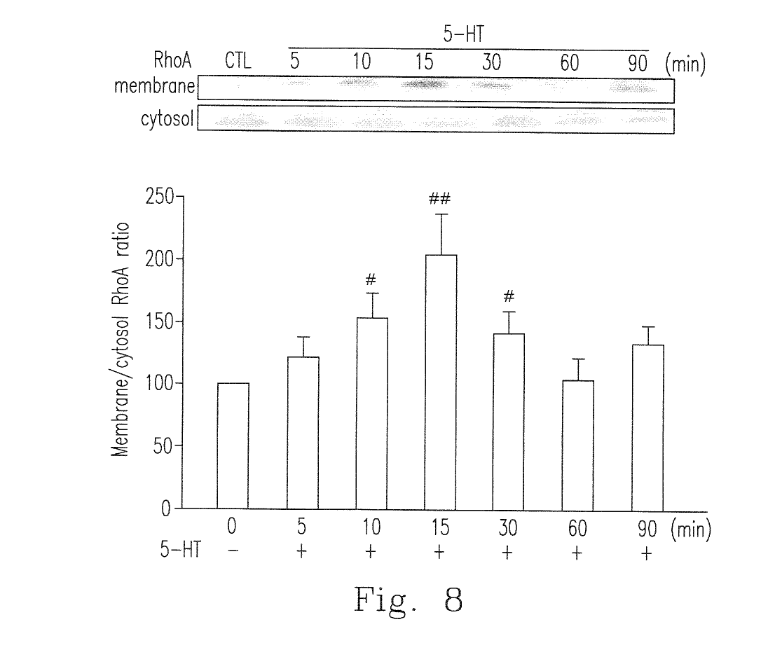

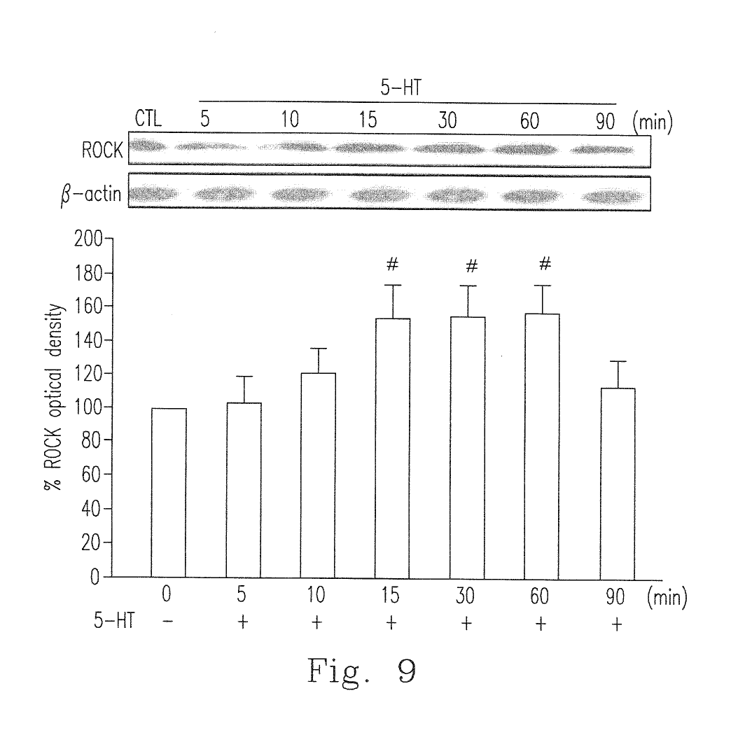

PASMCs were stimulated with 10 μM 5-HT for the indicated time periods. RhoA activity was analyzed as the membrane-to-cytosol ratio. Translocation of RhoA expression in the membrane extract was detected with anti-RhoA antibodies. 5-HT (10 μM) stimulated an increase in membrane/cytosol RhoA in PASMCs at 5 min, with a peak at 15 min and a return to basal levels by 60 min (FIG. 8). ROCK expression induced by 5-HT was significantly increased at 15-60 min and then returned to basal levels by 90 min (FIG. 9).

Attenuation of 5-HT-induced RhoA membrane localization and ROCK

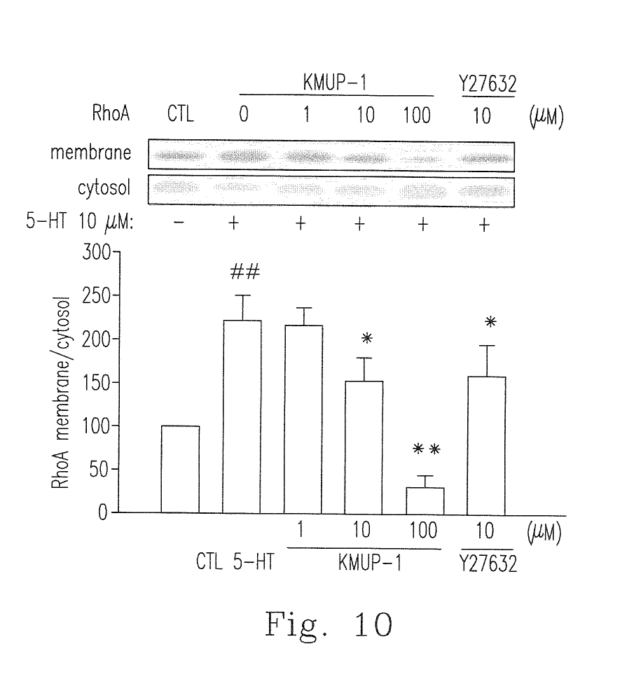

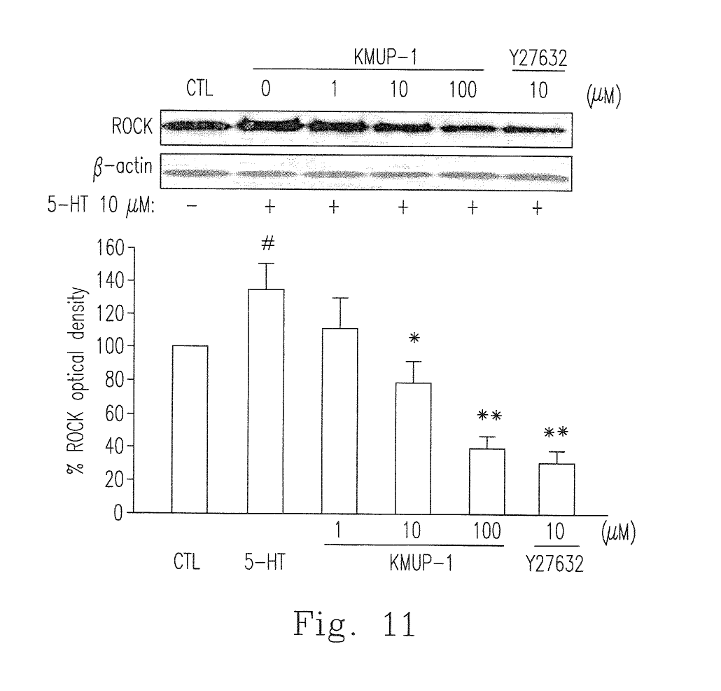

PASMCs were stimulated with 5-HT (10 μM) for 15 min prior to treatment of cells with different concentrations of KMUP-1 (1-100 μM) or Y27632 for 24 h. RhoA activation was examined by measuring the membrane-to-cytosol ratio. (FIG. 10), we examined the effect of KMUP-1 on 5-HT-induced ROCK. PASMCs were stimulated with 5-HT (10 μM) for 15 min prior to treatment of cells with different concentrations of KMUP-1 or Y27632 for 24 h. Treatment with KMUP-1 (1-100 μM) significantly inhibited 5-HT-induced ROCK (FIG. 11). Activation of RhoA is associated with its translocation from the cytosol to the membrane. Stimulation of PASMCs with 5-HT (10 μM, 15 min) led to an increased level of membrane-associated RhoA. 24 h treatment with KMUP-1 dose-dependently reversed the 5-HT-induced RhoA membrane association. Y27632 (10 μM) also inhibited the membrane-tocytosol ratio of RhoA and ROCK expression.

Attenuation of 5-HT-Induced Activation of ERK1/2 and AKT

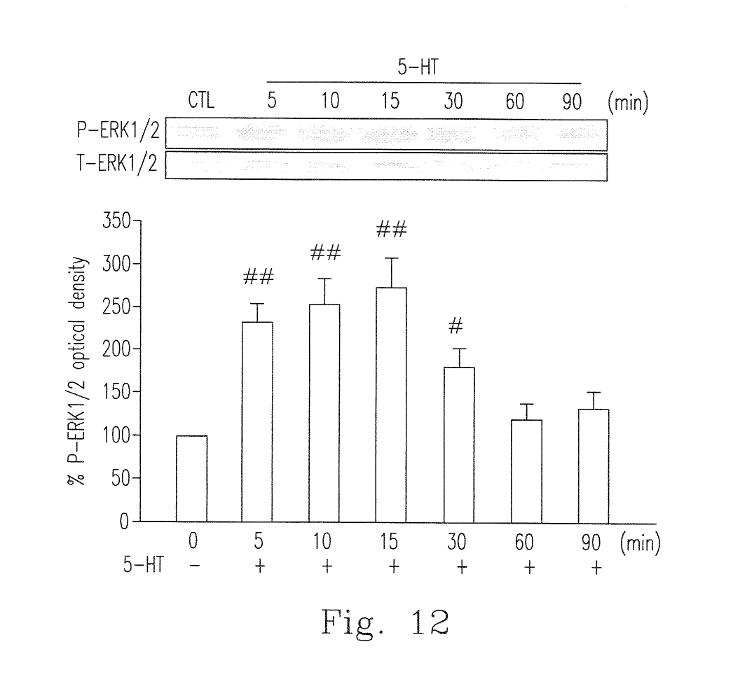

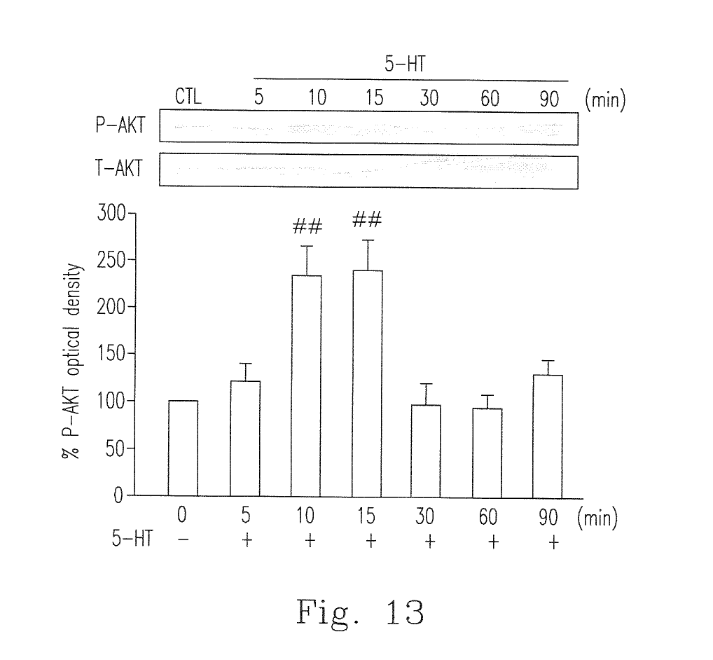

Phosphorylation or activation of ERK1/2 and AKT kinase is associated with proliferation in a variety of cell types, including PASMCs. Exposure of cells to 5-HT (10 μM) for 15 min (peak time for ERK1/2 and AKT phosphorylation) triggered 2.3-fold increases phosphorylation of ERK1/2 (FIG. 12) and AKT (FIG. 13).

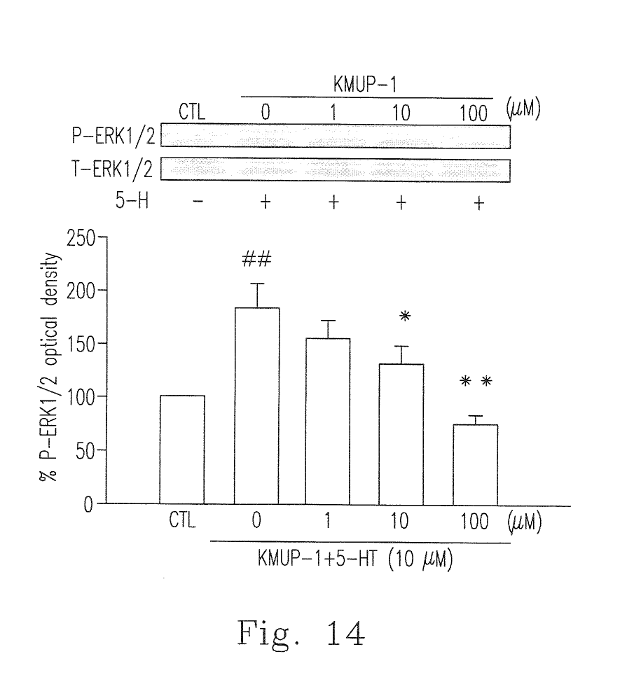

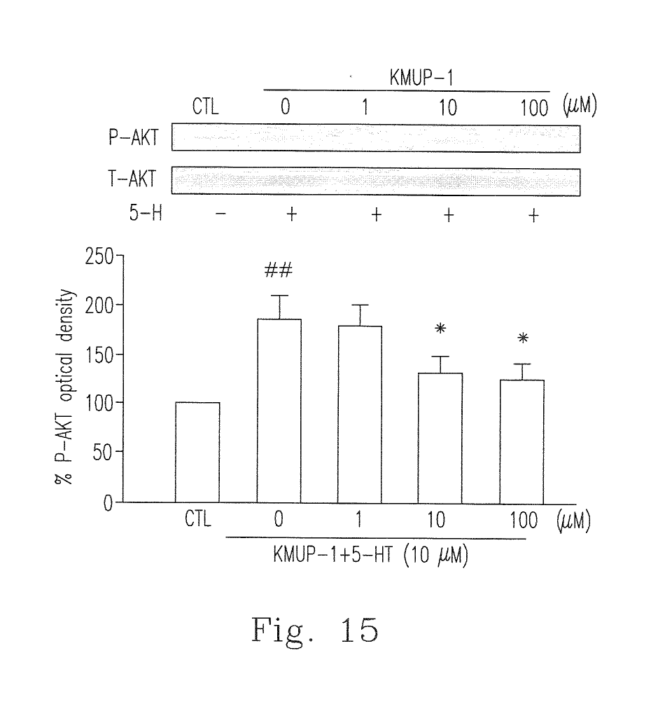

PASMCs were pre-incubated with KMUP-1 (1-100 μM) for 24 h and then stimulated with 5-HT (10 μM) for 15 min for phosphorylation of ERK1/2 and AKT. ERK1/2 and AKT phosphorylation were determined by Western blot analysis in whole cell lysates. Sample Western blots and the bar graph indicated that KMUP-1 concentration-dependently reduced 5-HT-stimulated ERK1/2 and AKT phosphorylation (n=4-6). This response was dose-dependently inhibited by pre-incubation of cells with KMUP-1 (1-100 μM) for 24 h (FIGS. 14 and 15).

Inhibition of 5-HT-Induced PASMCs Migration and Proliferation

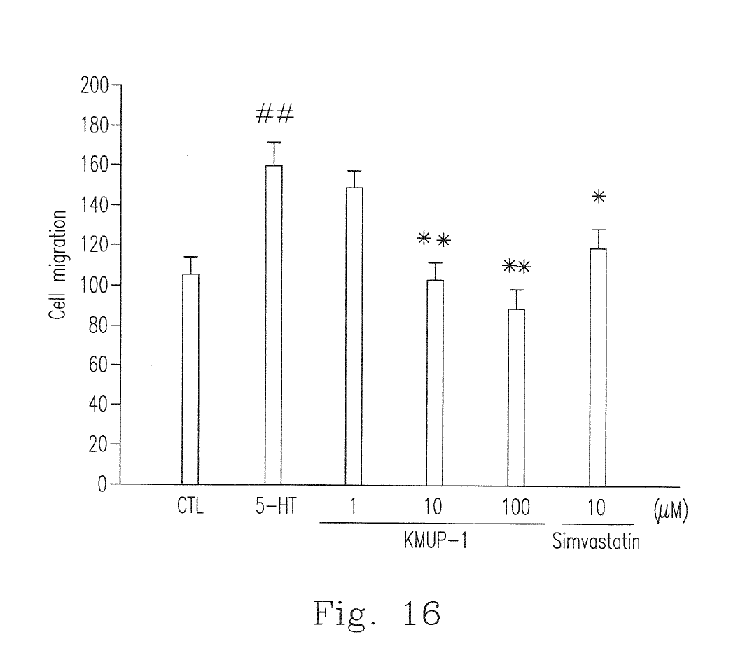

Quiescent PASMCs were pretreated with KMUP-1 (1-100 μM) and simvastatin (10 μM) for 20 min, then incubated with 5-HT (10 μM) for 24 h. Cell migration was measured by a cell wound healing assay as described in Methods. Phase contrast images were taken 24 h after wounding. Treatment with KMUP-1 (10-100 μM) and simvastatin (10 μM) concentration-dependently suppressed 5-HT-induced PASMCs migration. (FIG. 16).

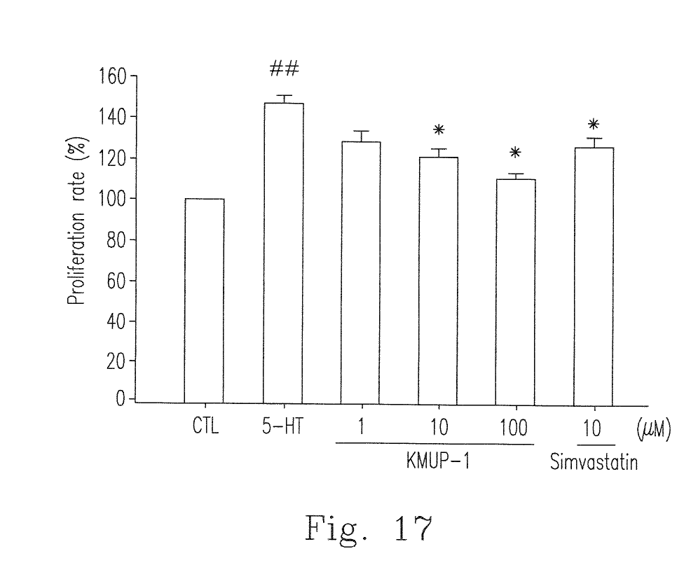

PASMCs proliferation, tested by MTT assay, was also dose-dependently inhibited by KMUP-1 and simvastatin (10 μM) cell proliferation was determined by MTT assay. KMUP-1 blocked 5-HT-induced PASMCs migration. Serum-starved PASMCs were stimulated with 10 μM 5-HT for 24 h with or without KMUP-1 (1-100 μM) or simvastatin (10 μM) pretreatment (FIG. 17).

TABLE 1
Estimated IC₅₀ and Kᵢ values of KMUP-1 in radioligand binding
to 5-HT_{2A}, 5-HT_{2B}, and 5-HT_{2C} in human recombinant CHO-K1 cells

| | | | Kᵢ values |
|---|---|---|---|
| | Radioligand | IC₅₀ [μM] | [μM] |
| 5-HT_{2A} | [³H]-ketanserin | 0.34 | 0.0971 |
| 5-HT_{2B} | [³H]-LSD | 0.04 | 0.0254 |
| 5-HT_{2C} | [³H]-mesulergine | 0.408 | 0.214 |

Radioligand binding on 5-HT_{2A}, 5-HT_{2B} and 5-HT_{2C} receptors

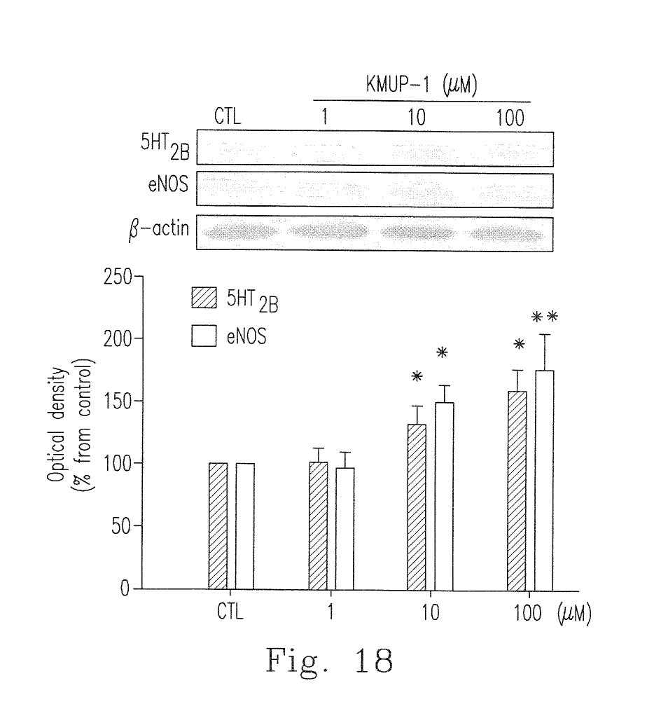

As shown in FIG. 18 and Table 1, the ligand specificity of the human 5-HT_{2B} receptor to KMUP-1 was more selective than the 5-HT_{2A} and 5-HT_{2C} receptors. The affinity of these sub-receptors for KMUP-1 is 5-HT_{2A}>5-HT_{2B}>5-HT_{2C}.

The expression of eNOS, 5-HT_{2B} receptor and the production of NO in Human Pulmonary Artery Endothelial Cells (HPAEC).

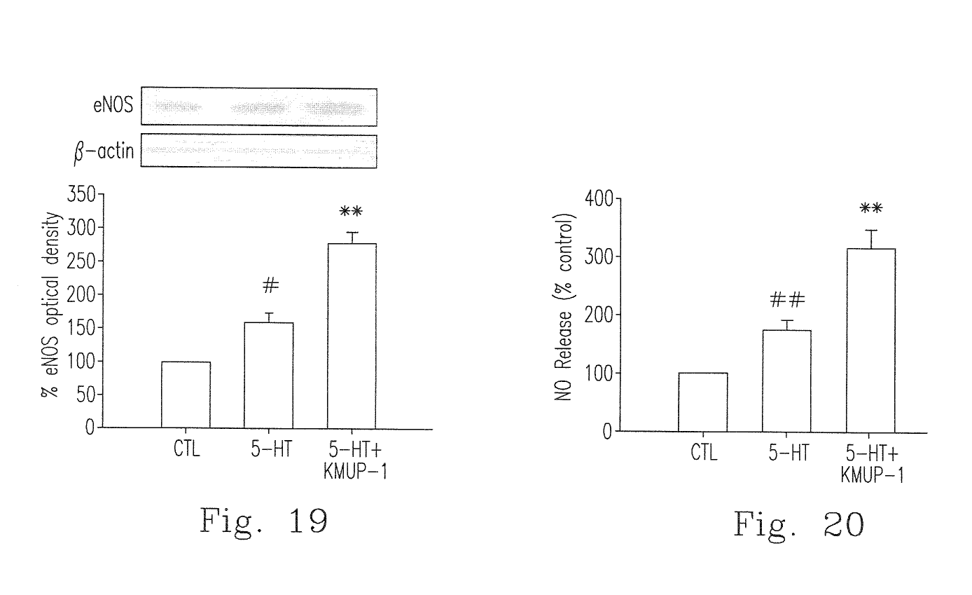

eNOS and 5-HT_{2B} receptor expressions were assessed by Western blotting. Incubation of HPAEC with KMUP-1 (1-100 μM) for 24 h dose-dependently increased eNOS and 5-HT_{2B} receptor expressions in cultured HPAEC (FIG. 19). Incubation of 5-HT (10 μM) for 30 min in HPAEC, 5-HT increased the expression of eNOS and the release of NO, compared to the control group. Treatment of HPAEC with KMUP-1 (10 μM) for 24 h before adding 5-HT also raised the expression of eNOS and the release of NO, compared to 5-HT treatment alone (FIG. 20).

TABLE 2
Changes of protein expression of eNOS, PDE-5A and ROCKII represented
by optical densitivity % after application of KMUPs salts (10 μM) for
120 min, compared to without treatment control

| Without treatment control | eNOS | PDE-5A | ROCKII |
|---|---|---|---|
| (Vehicle) | 100 (%) | 100 (%) | 100 (%) |
| KMUP-1 HCl | 155 ± 14.8 | 64 ± 4.5 | 42 ± 6.3 |
| KMUP-1-citric acid | 152 ± 13.6 | 66 ± 5.2 | 43 ± 3.8 |
| KMUP-1-nicotinic acid | 158 ± 12.4 | 62 ± 4.8 | 41 ± 2.5 |
| KMUP-2 HCl | 147 ± 8.6 | 68 ± 5.3 | 40 ± 3.7 |
| KMUP-2-citric acid | 145 ± 7.2 | 71 ± 5.4 | 40 ± 3.7 |
| KMUP-3 HCl | 148 ± 7.5 | 63 ± 5.2 | 44 ± 2.9 |
| KMUP-3-nicotinic acid | 145 ± 7.5 | 60 ± 4.1 | 43 ± 1.6 |
| KMUP-4 HCl | 135 ± 7.5 | 73 ± 3.4 | 37 ± 2.8 |
| KMUP-4-citric acid | 131 ± 6.7 | 78 ± 3.6 | 56 ± 3.4 |

P < 0.05; significantly different from control (n = 5)

Matrix metalloproteinases (MMPs) are a large family of endopeptidases that degrade components of the extracellular matrix in vascular and airway system. Of the MMPs family, MMP-9 is involved in extracellular matrix turnover because of its ability to cleave proteins constituting the extracellular matrix. Notably, MMP-9 induces migration of eosinophils, lymphocytes and neutrophils across basement membranes in response to inflammatory mediators.

Very late antigen-4 (VLA-4), a counter-receptor of vascular cell adhesion molecule-1 (VCAM-1) on endothelium, plays an important role in the migration of eosinophils and lymphocytes to inflammation sites. Lymphocyte function associated antigen (LFA-1) on neutrophils and monocytes interacts with intercellular-associated molecule (ICAM-1), a counter receptor for LFA-1 on endothelium in inflamed areas. Inhibition of VCAM-1/ICAM-1 expression has the potential to interfere with migration of eosinophils and lymphocytes. In this regard, MMP inhibitor regulates inflammatory cell migration by reducing ICAM-1 and VCAM-1 expression.

Pulmonary e-NOS Enhancement and MMP-9 Suppression

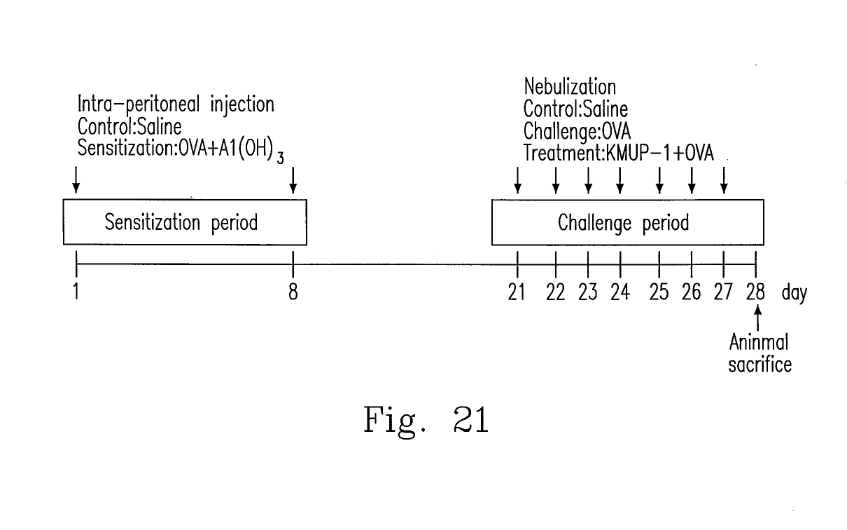

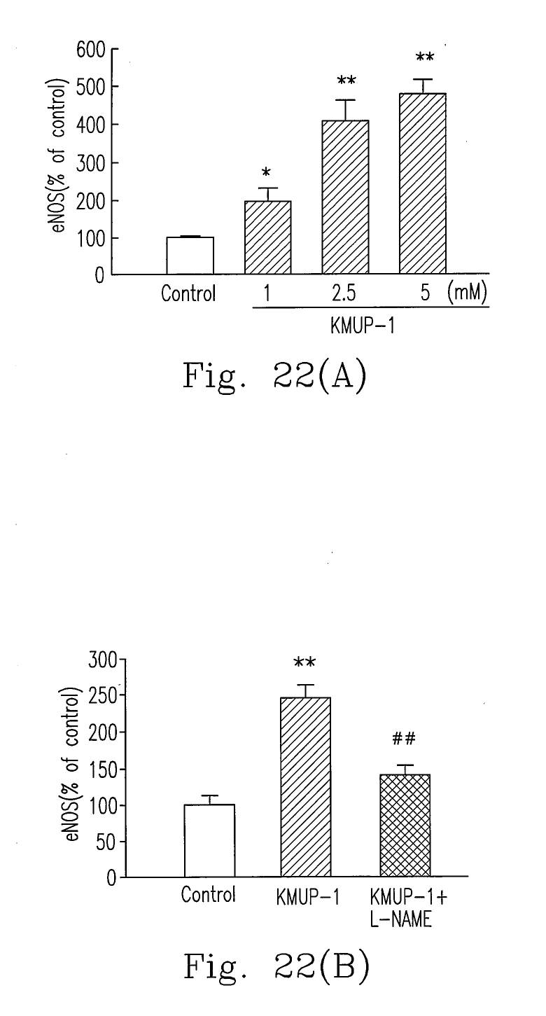

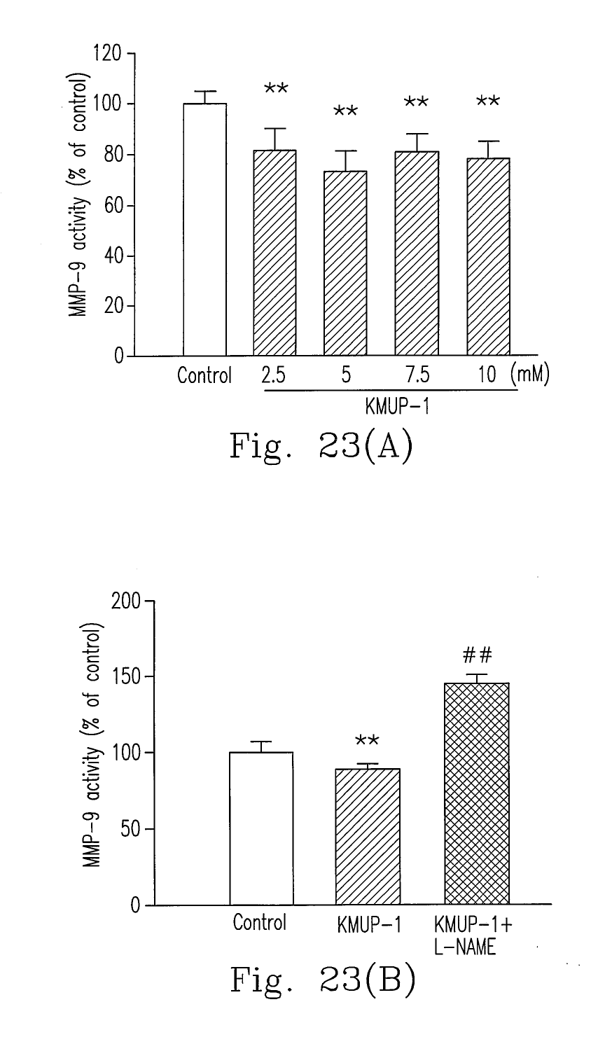

In the short-term experiment without ovalbumine sensitization (FIG. 21), KMUP-1 (1.0˜2.5 mM, 30 mins) dose-dependently increased eNOS and decreased MMP-9 expression in mice lung tissues; the changes of eNOS and MMP-9 expression by KMUP-1 (2.5 mM, 30 mins) were reversed by L-NAME-pretreatment (12 mM, 30 mins) (FIG. 22A, 22B, 23A, 23B; *P<0.05 versus control; “P<0.01 versus control, ##P<0.01 versus KMUP-1).

Pulmonary Vascular Wall Thickening Features

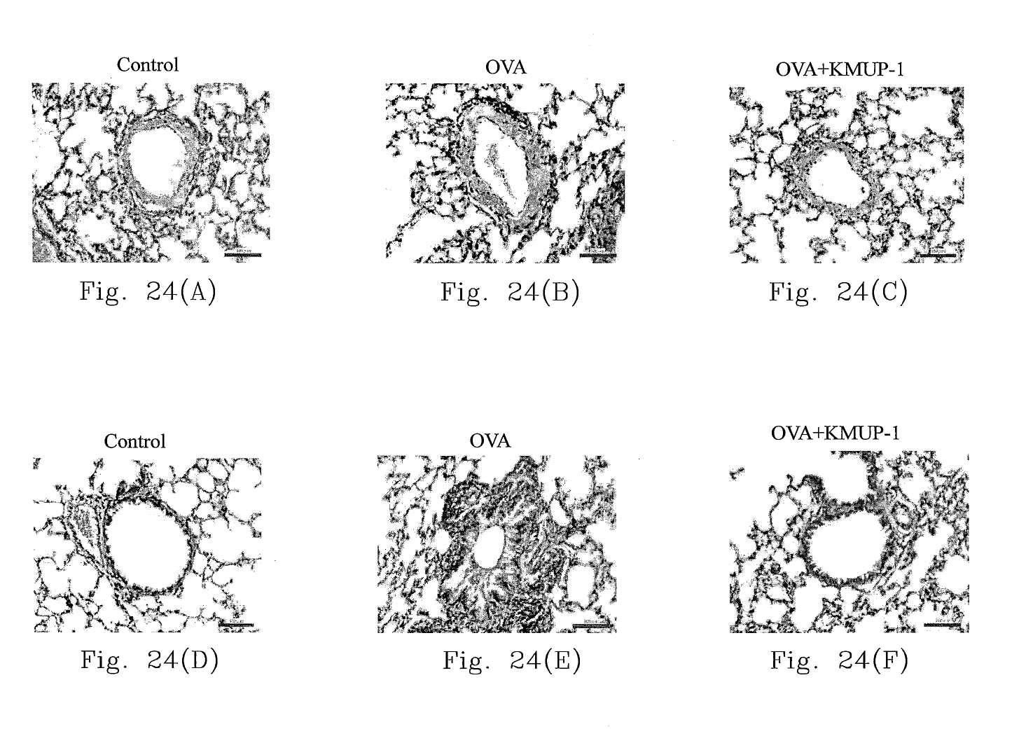

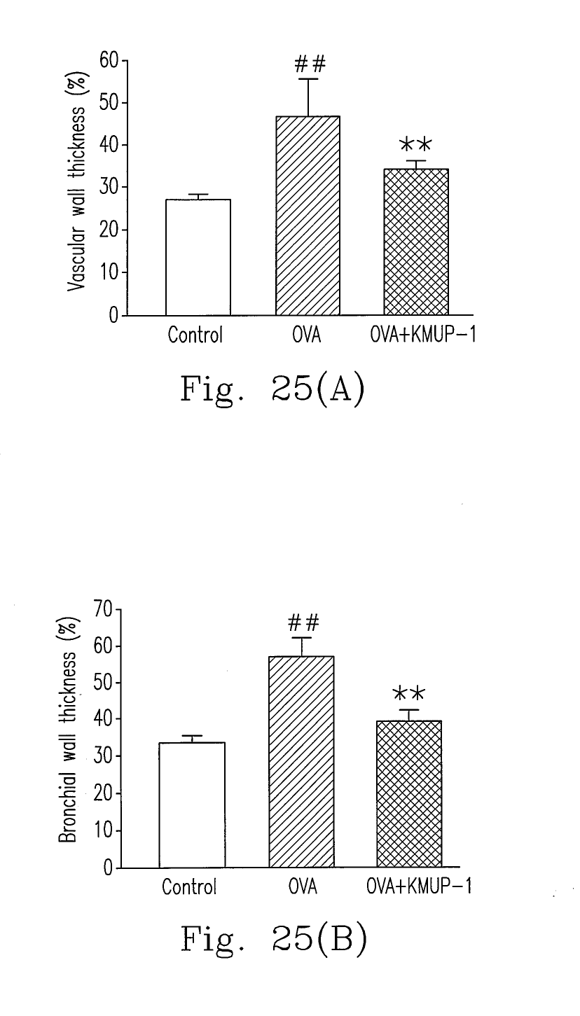

Histological analyses revealed typical pathologic features of asthma in the ovalbumine-exposed mice. Numerous inflammatory cells including eosinophils were infiltrated around the peri-vascular and peri-bronchial region (FIGS. 24B, 24E), compared to control mice (FIGS. 24A, 24D). Mice treated with KMUP-1 showed marked reductions of inflammatory cells in the peri-vascular (FIG. 24C) and peri-bronchiolar regions (FIG. 24F). After ovalbumine challenge, marked increases in PA wall thickness % were found, compared to controls (FIG. 25A, ##P<0.01 versus control; **P<0.01 versus ovalbumine).

Pulmonary eNOS, iNOS, sGCα1, PKG and MMP-9 Expression

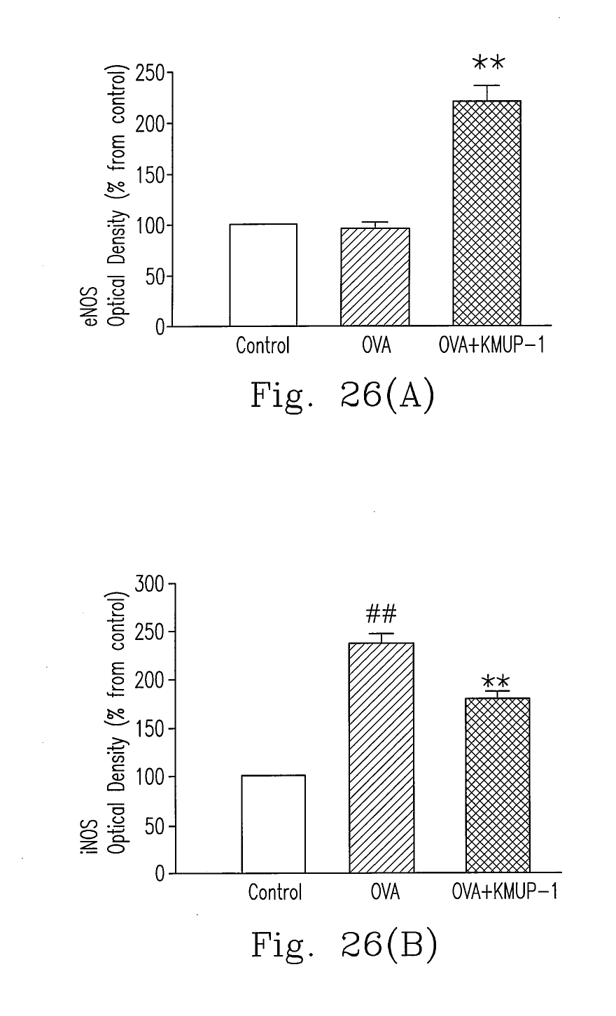

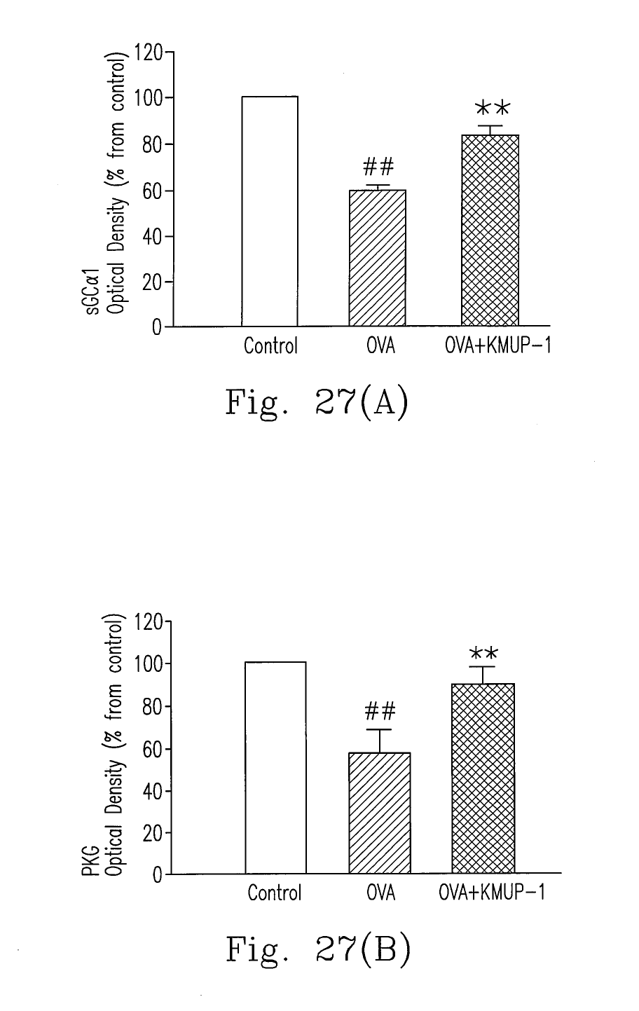

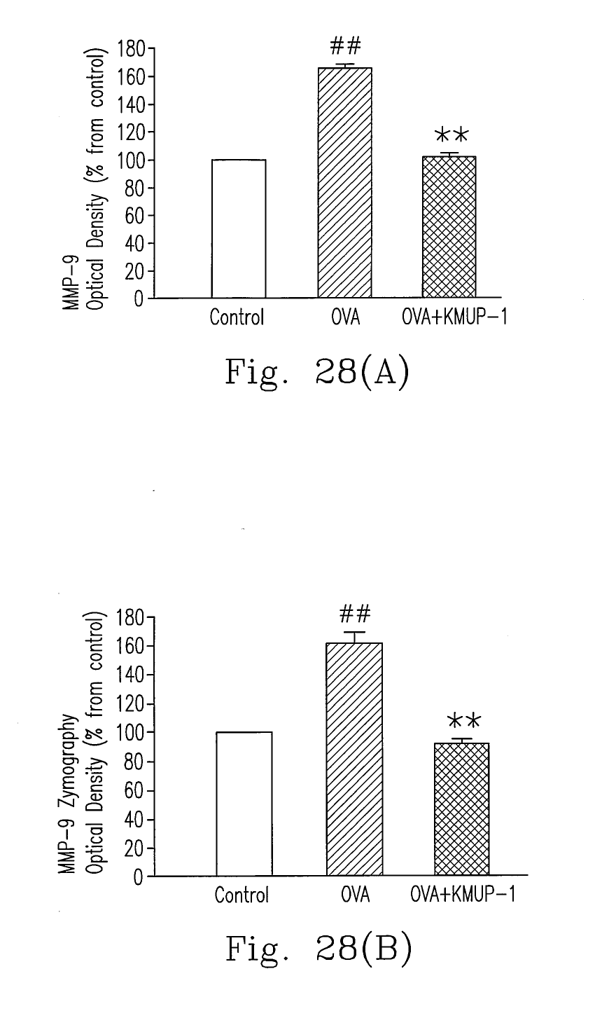

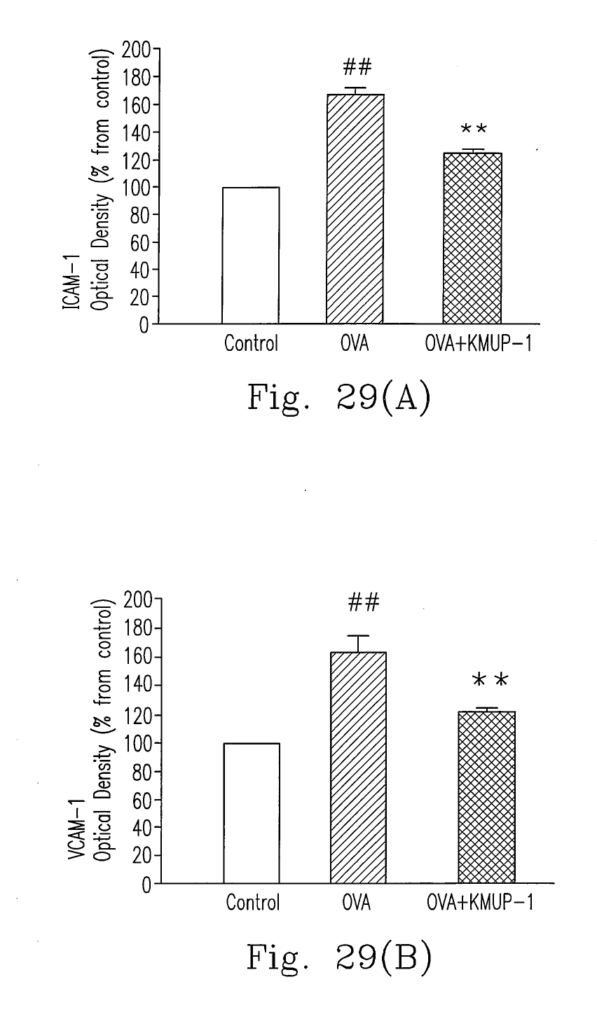

In the 28-day experiment, Western blotting analysis showed that the levels of basal eNOS expression were not affected by ovalbumine, but iNOS was increased and sGCα1, PKG, MMP-9, ICAM and VCAM-1 were decreased significantly at 24 hrs after the last ovalbumine-challenge. The increased iNOS and MMP-9 and decreased sGCα1 and PKG expressions by ovalbumine sensitization were significantly reduced by nebulization of KMUP-1 (FIGS. 26A, 26B, 27A, 27B, 28A, 28B, 29A and 29B; ##P<0.01 versus control; **P<0.01 versus OVA). Both expression and its zymography of MMP-9 were decreased (FIGS. 28A and 28B; **P<0.01 versus ovalbumine).

Immunostaining

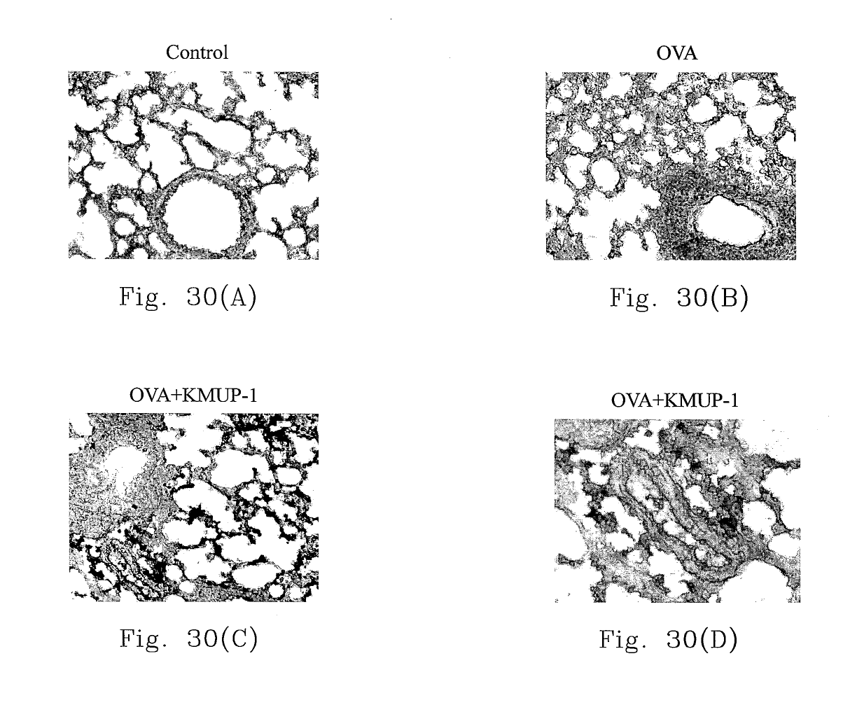

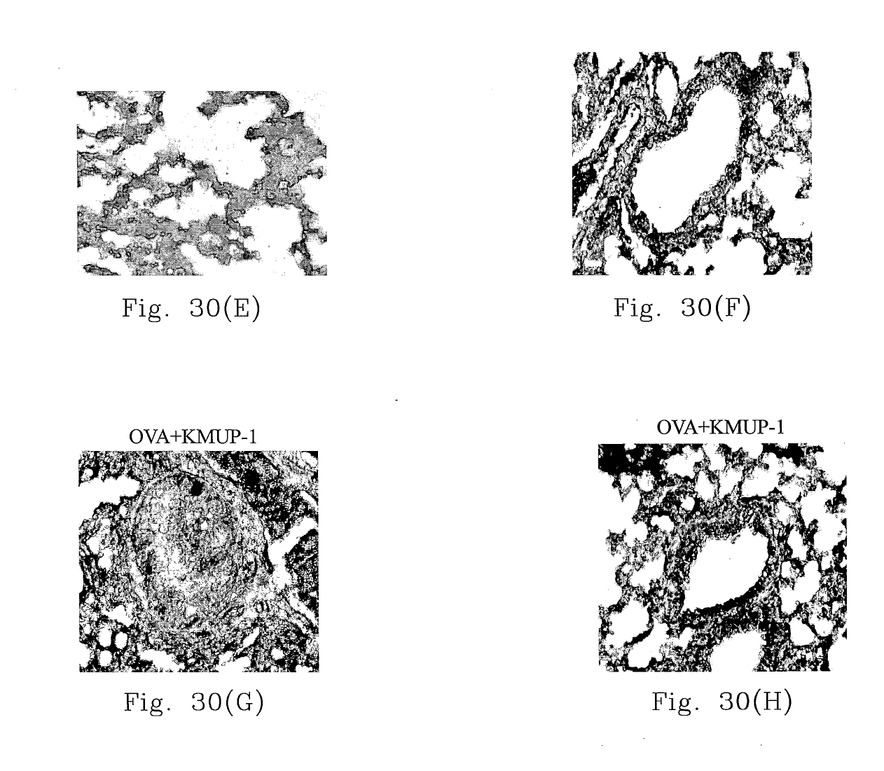

ovalbumine decreased vascular eNOS-immunostaining in lung sections, but KMUP-1 prevented this decrease quantitatively (FIGS. 30A, 30B, 30C, 30D and 30E). Immunostaining also indicated that MMP-9 was found on inflammatory cells and debris, filling the airway lumen (FIG. 30G). In control and ovalbumine-sensitized and ovalbumine-challenged mice treated with KMUP-1, MMP-9 positive cells were not found and hardly detected, respectively (FIGS. 30G, 30H).

NOx Levels and Inflammatory Cell Components

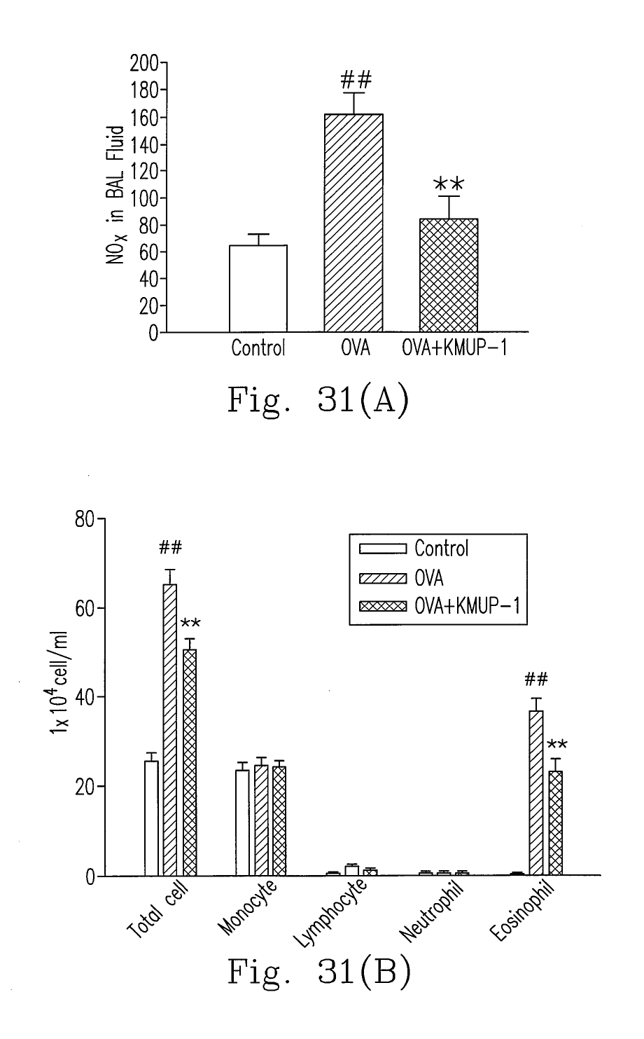

Griess reagent analysis showed that the basal levels of NOx (65.4±8.2 μM) in BALF were significantly increased to 162.8±15.7 μM by ovalbumine at 24 hrs after the last challenge, compared with levels after saline nebulization. The increased levels of NOx (nitrate+nitrite) in BALF were significantly reduced by KMUP-1 to 86.3±17.3 μM (FIG. 31A; ##P<0.01 versus control; **P<0.01 versus ovalbumine).

The numbers of total cells, lymphocytes and eosinophils in BALFF were significantly increased at 24 hrs after ovalbumine challenge. KMUP-1 nebulization significantly reduced the increase in total cells, lymphocytes and eosinophils elicited in the airway lumen 24 hrs after the last ovalbumine challenge (FIG. 31B; ##P<0.01, versus control; **P<0.01 versus ovalbumine).

Plasma Cytokines

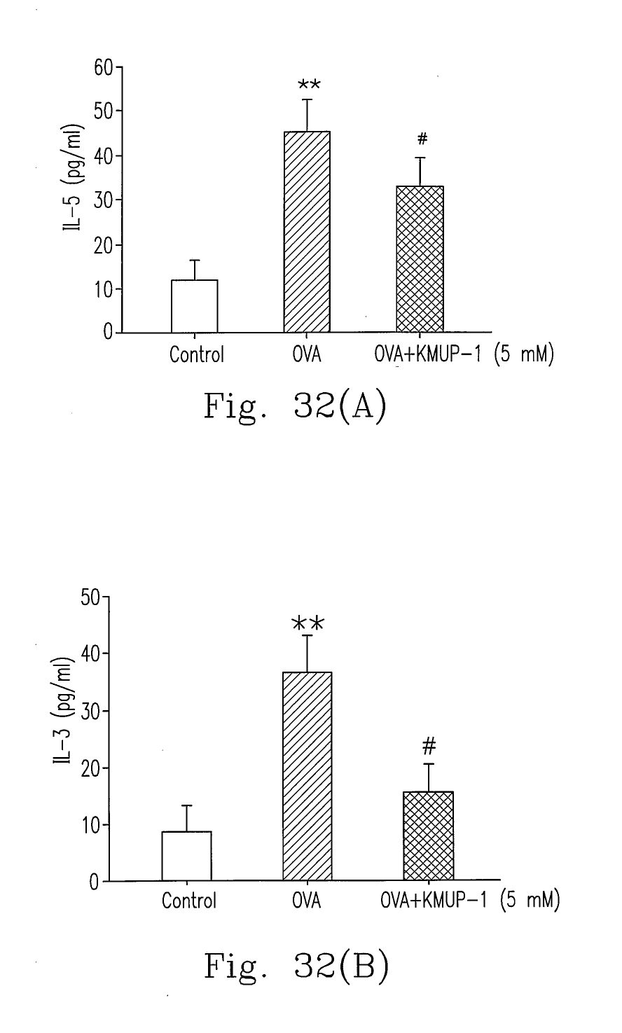

FIG. 32 shows that ovalbumine increased IL-5 (A) and IL-13 (B), respectively and KMUP-1 inhibited IL-5 and IL-13 levels in the plasma of mice sensitized and challenged by ovalbumine (**P<0.01 versus control; #P<0.05 versus ovalbumine).

Table 3 shows the estimated IL-5 and IL-13 values in the plasma of mice sensitized and challenged by ovalbumine (OVA).

TABLE 3

| | IL-5 (pg/ml) | IL-13 (pg/ml) |
|---|---|---|
| Without treatment control | 11 ± 3 | 9 ± 4 |
| (Vehicle) | | |
| OVA-sens | 43 ± 6 | 35 ± 6 |
| +KMUP-1-HCl (Inhal) | 31 ± 6 | 14 ± 5 |
| 5 mM OVA-sens | | |
| +KMUP-5 (Inhal) 5 mM | 29 ± 4 | 12 ± 3 |
| OVA-sens | | |
| +Simvastatin (oral) 2.5 mg Kg⁻¹ | 158 ± 12.4 | 41 ± 2.5 |
| OVA-sens | | |
| +KMUP-2 HCl(Inhal) 5 mM | 32 ± 6 | 15 ± 3 |
| OVA-sens | | |
| +KMUP-1 HCl (Inhal) 5 mM | 28 ± 4 | 14 ± 2 |
| +Simvastatin (oral) 2.5 mg Kg⁻¹ | | |
| OVA-sens | | |

OVA-sens = ovalbumine-sensitization

Table 4 illustrates examples of some combination therapies of the present invention wherein the combination comprises an effective amount of a KMUPS derivative compound and an additional active agent of a statins analogues, wherein said combination together comprises a pulmonary disease inhibiting effective amount of said compounds.

TABLE 4

| Without treatment control; non- | iNOS | MMP-9 |
|---|---|---|
| treatment allergic animal (Vehicle) | 100 (%) | 100 (%) |
| KMUP-1 HCl (Inhal) 5 mM | 55 ± 14.8 | 42 ± 6.3 |
| KMUP-1 HCl (Inhal) 5 mM | 52 ± 13.6 | 43 ± 3.8 |
| KMUP-5 (Inhal) 5 mM | | |
| Simvastatin (oral) 2.5 mg Kg⁻¹ | 58 ± 12.4 | 41 ± 2.5 |
| KMUP-2 HCl (Inhal) 5 mM | 47 ± 8.6 | 40 ± 3.7 |
| KMUP-2 HCl (Inhal) 5 mM | 45 ± 7.2 | 40 ± 3.7 |
| Simvastatin (oral) 2.5 mg Kg⁻¹ | | |
| KMUP-3 HCl (Inhal) 5 mM | 48 ± 7.5 | 44 ± 2.9 |
| KMUP-3 HCl (oral) 2.5 mg Kg⁻¹ | 45 ± 7.5 | 43 ± 1.6 |
| KMUP-4 HCl (Inhal) 5 mM | 35 ± 7.5 | 37 ± 2.8 |
| KMUP-4 HCl (oral) 2.5 mg Kg⁻¹ | 31 ± 6.7 | 56 ± 3.4 |

P < 0.05; significantly different from control (n = 5)
Inhal = inhalation administration
Oral = oral administration
KMUP-5 = synthesized KMUP-1-Simvastatin compound

Table 5 illustrates examples of some combination therapies of the present invention wherein the combination comprises an effective amount of a KMUPS derivative compound and an additional active agent of a statins analogues, wherein said combination together comprises the white blood cell total amounts, e.g. neutrophils, lymphocytes and eosinophils elicited in the airway lumen 24 hrs after the last ovalbumine challenge.

TABLE 5

| | Eosinophil | Lymphocyte | Neurophil |
|---|---|---|---|
| Without treatment control; | 0.3 ± 0.1 | 0.7 ± 0.2 | 0.9 ± 0.2 |
| non-treatment allergic animal | | | |
| (Vehicle) | | | |
| OVA | 37.2 ± 2.8 | 2.4 ± 0.3 | 1.0 ± 0.2 |
| KMUP-1 HCl (Inhal) 5 mM | 23.7 ± 2.8 | 1.5 ± 0.3 | 1.0 ± 0.2 |
| KMUP-5 (Inhal) 5 mM | 22 ± 2.4 | 1.3 ± 0.5 | 1.0 ± 0.3 |
| Simvastatin (oral) 2.5 mg Kg⁻¹ | 27 ± 1.6 | 1.8 ± 0.7 | 1.5 ± 0.4 |
| KMUP-2 HCl(Inhal) 5 mM | 25 ± 2.2 | 1.2 ± 3.7 | 1.0 ± 0.4 |
| KMUP-2 HCl (Inhal) 5 mM | 20 ± 3.5 | 1.4 ± 0.6 | 1.0 ± 0.2 |
| Simvastatin (oral) 2.5 mg Kg⁻¹ | | | |
| KMUP-3 HCl (Inhal) 5 mM | 24 ± 3.5 | 1.3 ± 0.6 | 1.0 ± 0.3 |
| KMUP-3 HCl (oral) 2.5 mg | 25 ± 2.5 | 1.7 ± 2.8 | 1.1 ± 0.3 |
| Kg⁻¹ | | | |
| KMUP-4 HCl (Inhal) 5 mM | 21 ± 6.7 | 1.6 ± 3.4 | 1.1 ± 0.2 |
| KMUP-4 HCl (oral) 2.5 mg | 20 ± 3.5 | 1.7 ± 1.8 | 1.1 ± 0.3 |
| Kg⁻¹ | | | |
| | | monocyte | Total cells |
| Without treatment control; | 0.3 ± 0.1 | 0.7 ± 0.2 | 25.6 ± 1.8 |
| non-treatment allergic animal | | | |
| (Vehicle) | | | |
| OVA | 37.2 ± 2.8 | 2.4 ± 0.3 | 65.3 ± 3.4 |
| KMUP-1 HCl (Inhal) 5 mM | 23.7 ± 2.8 | 1.5 ± 0.3 | 50.6 ± 2.4 |
| KMUP-5 (Inhal) 5 mM | 22 ± 2.4 | 1.3 ± 0.5 | 48.2 ± 0.5 |
| Simvastatin (oral) 2.5 mg Kg⁻¹ | 27 ± 1.6 | 1.8 ± 0.7 | 52.5 ± 0.4 |
| KMUP-2 HCl(Inhal) 5 mM | 25 ± 2.2 | 1.2 ± 3.7 | 51.0 ± 0.4 |
| KMUP-2 HCl (Inhal) 5 mM | 20 ± 3.5 | 1.4 ± 0.6 | 47.0 ± 0.3 |
| Simvastatin (oral) 2.5 mg Kg⁻¹ | | | |
| KMUP-3 HCl (Inhal) 5 mM | 24 ± 3.5 | 1.3 ± 0.6 | 52.1 ± 0.2 |
| KMUP-3 HCl (oral) 2.5 mg | 25 ± 2.5 | 1.7 ± 2.8 | 51.2 ± 0.3 |
| Kg⁻¹ | | | |
| KMUP-4 HCl (Inhal) 5 mM | 21 ± 6.7 | 1.6 ± 3.4 | 53.4 ± 0.3 |
| KMUP-4 HCl (oral) 2.5 mg | 20 ± 3.5 | 1.7 ± 1.8 | 51.8 ± 0.4 |
| Kg⁻¹ | | | |

P < 0.05; significantly different from control (n = 5)
Inhal = inhalation administration
Oral = oral administration
KMUP-5 = synthesized KMUP-1-Simvastatin compound Number × 10⁴ cell/ml

### Biological Experiments

Animal

Care and use of experimental animals (BALB/c mice) followed the Guide for the Care and Use of Laboratory Animals published by the US National Institutes of Health (NIH Publication No. 85-23, 1996). The experimental protocol was approved by the animal center of Kaohsiung Medical University in Taiwan.

Animal models and hemodynamic measurement

The experiments were performed in adult male Wistar rats (300 to 350 g) in accordance with institutional guidelines after approval by the ethical review committee. PAH development and pulmonary expression of 5-HTT were examined in rats after 60 mg/kg single injection (i.p.) of monocrotaline (MCT). To assess the potential preventive effect of KMUP-1 on MCT-induced PAH and associated proliferation, we assigned rats at random to 2 groups of 8 animals which received KMUP-1 at 5 mg/kg/day p.o or 1 mg/kg/day i.p. All treatments were given once a day for 3 weeks after a single MCT injection (60 mg/kg i.p.) (Abe et al., 2004). On day 21, rats were anesthetized and pulmonary artery blood pressure (MPAP) was recorded as previously described. Lung tissues were dissected for Western blotting and immunohistochemistry.

Anti-RhoA monoclonal antibody and anti-ERK1/2 rabbit antibody were purchased from Santa Cruz Biotechnology (CA, USA). Anti-5-HT_{2B}, anti-eNOS and anti-ROCK (ROCKII) antibody were purchased from Upstate Biotechnology (Lake Placid, N.Y., USA). Anti-5-HTT rabbit antibody was purchased from Chemicon Biotechnology (Temecula, Calif., USA). Anti-phosphor-ERK1/2, anti-AKT, anti-phospho-AKT, and horseradish peroxidase-conjugated polyclonal rabbit and mouse antibody were purchased from Santa Cruz Biotechnology. Anti-PKG; anti-VCAM and anti-ICAM-1 antibodies from Santa Cruz (CA, USA); anti-sGCα1, anti-MMP-9, ROCKII (a representative of Rho kinase), iNOS, IL-5, IL-13, and β-actin antibodies from Sigma Chemical Co. Goat anti-rabbit IgG horseradish peroxidase conjugated secondary antibody was purchased from Santa Cruz (CA, USA). [³H]mesulergine was purchased from Amersham (Buckinghamshire, UK). [³H] ketanserin was purchased from Perkin-Elmer (Shelton, Conn., USA).

KMUPS derivative was synthesized in our laboratory and whose HCl salt was dissolved in vehicle and the serial dilutions was made in distilled water for the experiments of inhalation. All other reagents we used were of analytical grade or higher and were obtained from commercial sources.

Measurement of Plasma 5-HT Levels

After MCT-treatment, rats acquired severe PAH and received sodium pentobarbital (40 mg/kg, i.p.) at day 21 for surgical anesthesia and measurement of MPAP (Chung et al., 2010). Blood samples (1.0 mL) were obtained from the heart. Blood was transferred to plastic tubes that included EDTA and was centrifuged at 100 g for 20 min. The plasma was transferred to tubes and stored at −80° C. until analysis. Plasma 5-HT levels were determined using commercially available Serotonin EIA (IBL, Minneapolis, USA). Cross-reactivity with related substances (e.g., 5-HIAA, phenylalanine, histidine and tyramine) has been reported to be <0.002%. Results were read from a standard curve. The threshold of detection was 0.3 ng/mL.

Isometric Force of Pulmonary Artery

Wistar rats were euthanized with an overdose of sodium pentobarbital (60 mg/kg, i.p.) before open-chest surgery. During surgery, a thoracic retractor was used to help isolate the pulmonary artery. The chest was opened to dissect the second branches of the main pulmonary artery, which were cut into 2-3 mm rings, suspended under isometric conditions and connected to a force transducer as previously described (Ugo Basile, Model 7004, Comerio-VA, Italy) to measure the constriction caused by 5-HT (10 μM). The pulmonary artery ring preparations were stretched to a basal tension of 1 g and allowed to equilibrate for 60-90 min. After equilibration, pulmonary artery rings were constricted with 5-HT (10 μM) to prime the tissues and check the functionality of the endothelium (at least 80% relaxation in response to acetylcholine 1 μM). Once the contractile response to each agonist reached a stable tension, KMUP-1 (0.1-100 μM) was cumulatively added to the organ bath in the presence of 5-HT (10 μM, pre-incubation time of 15 min). The effect of KMUP-1 on NOS and 5-HT was also studied in vessels pre-incubated with the NOS inhibitor L-NAME (100 μM) 20 min before 5-HT administration. The percentage of relaxation was estimated using the following equation: relaxation (%)=(maximal contraction−relaxation level)/(maximal contraction-basal level)×100. Data was obtained from serotonin-induced maximal contractile responses in pulmonary artery.

PASMCs Proliferation in MCT-Treated Lung Tissues

We evaluated proliferating cell nuclear antigen (PCNA) to assess PASMCs proliferation in rats treated with MCT alone or with KMUP-1. Lung tissue sections were de-paraffinized in xylene and then treated with a graded series of alcohol washes, rehydrated in PBS (pH 7.5), and incubated with target retrieval solution (DAKO Co, Tokyo, Japan) in a water bath at 90° C. for 20 min. Endogenous peroxidase activity was blocked with H₂O₂ in PBS (3%, vol/vol) for 5 min. Slides were then washed in PBS, incubated for 30 min in a protein blocking solution, and incubated for 30 min with anti-PCNA mouse monoclonal antibody (PC-10, 1:200, Dako). Antibodies were washed off, and the slides were processed with an alkaline phosphatase LSAB and system horseradish peroxidase detection kit (DAKO Co, Tokyo, Japan). Brown color was generated with a DAB substrate, and nuclei were counter-stained with hematoxylin.

Lung 5-HTT Immunohistochemical Analysis

For 5-HTT immunostaining, the lung slides were dewaxed in 100% xylene, and the sections were then rehydrated by successive immersion first in decreasing ethanol concentrations (100%, 90%, 80%, 50% and 30%) and then in water. Endogenous peroxidase activity was blocked using H₂O₂ in methanol (0.3% vol/vol) for 10 min. After three PBS washes, sections were pre-incubated in PBS supplemented with 3% (wt/vol) BSA for 30 min, then incubated overnight at 4° C. with goat polyclonal anti-5-HTT antibody (Abcam Biotechnology, Cambridge, UK) diluted to 1:1000 in 1×PBS, 0.02% BSA. Next, the sections were exposed for 1 h to biotin-labeled anti-goat secondary antibodies (DAKO Co, Tokyo, Japan) diluted 1:1000 in the same buffer. Peroxidase staining of the slides incubated in streptavidin-biotin horseradish peroxidase solution was carried out using 3,3′-diaminobenzidine tetrahydrochloride dihydrate (DAB; DAKO Co, Tokyo, Japan) and hydrogen peroxide. Finally, the sections were stained with hematoxylin and eosin.

Preparation of PASMCs

Wistar rats were anesthetized with an overdose of sodium pentobarbital (60 mg/kg) and the skin was sterilized with 75% alcohol. The chest was opened and the heart and lung were removed. The organs were rinsed several times in PBS. The pulmonary artery were segregated in a sterile manner. The outer sphere was peeled and the microtubule was snipped visually, and endothecia were shaved lightly 2-3 times in order to remove endothelial cells. The tunica media was prepared into scraps (1 mm³) in DMEM (Dulbecco's modified Eagle's medium). PASMCs were cultured in DMEM containing 10% fetal bovine serum (5% CO₂ at 37° C.). The culture medium was changed every 3 days and cells were subcultured until confluence. Primary cultures of 2-4 passages were used in the experiments. Cells were examined by immunofluorescence staining of α-actin to confirm the purity of PASMCs. Over 95% of the cell preparations were found to be composed of smooth muscle cells.

Human Pulmonary Artery Endothelial Cells (HPAEC) Cultured

HPAEC purchased from ATCC were maintained in humidified incubator containing 5% CO₂ at 37° C. HPAEC were cultured in F-12 supplemented with 10% fetal bovine serum. The culture medium was changed every 3 days and cells were subcultured until confluence. HPAEC were harvested with a solution of trypsin-EDTA (GIBCO BRL, NY, USA) while in a logarithmic phase of growth. HPAEC were used between passages 4-8.

Microculture Tetrazolium Test (MTT)

PASMCs were seeded into 96-well plates at a density of 1×10⁴ cells/well. The cells were then incubated in medium containing vehicle (1% FBS DMEM) and 5-HT (10 μmol/L) for 24 h with or without KMUP-1 (1-100 μM) and simvastatin (10 μM) added 30 min before 5-HT. At the end of this period, MTT (2 g/L) was added to each well, and incubation proceeded at 37° C. for 4 h. Thereafter, the medium was removed and the cells were solubilized in 150 μL DMSO. The optical density (OD) of each well was determined by enzyme-linked ELISA at 540 nm of wavelength.

PASMCs [Ca²⁺]i Measurement

The measurement of [Ca²⁺]i in PASMCs was performed using a spectrofluorophotometer as previously reported. PASMCs, cultured for 2-4 passages and re-suspended by trypsin, were loaded with Fura-2/AM for the measurement of [Ca²⁺]i changes in cells within the cuvette by spectrofluorophotometer (Shimadzu, RF-5301PC, Shimadzu, Japan). KMUP-1 was applied 5 min before application of 5-HT (10 μM).

Western Blotting

PASMCs were stimulated with 5-HT or not, as indicated. Cells were treated with KMUP-1 for 24 h before 5-HT stimulation. Whole lysates were collected and resolved by SDS-PAGE as previously described. Primary antibodies were anti-β-actin at: 1:10,000 dilution and anti-RhoA, anti-ROCK, antiphospho-ERK1/2, anti-phospho-AKT, anti-ERK1/2, anti-AKT, anti-5HTT, anti-5-HT_{2B} and anti-eNOS at 1:1000. All blots were incubated with antibodies at 4° C. overnight. After being washed, the appropriate secondary antibodies were added at a dilution of 1:1000 for 1 h at room temperature. After extensive washing, blots were developed with a Super Signal enhanced chemiluminescence kit (Biorad, CA, USA) and visualized on Kodak AR film.

RhoA Translocation

Previous reports have shown that the active form of RhoA is translocated from the cytosol to the plasma membrane, where it activates Rho kinase. Therefore, the membrane/cytosol ratio of RhoA is considered a measure of RhoA activity. The cytosol and membrane protein of PASMCs were extracted with a CNM (cytosol, nuclear, membrane) kit. To assess membrane trans-location of RhoA, protein in membrane and cytosolic fractions was determined by standard Western blot analysis using mouse monoclonal anti-RhoA antibody (1:1000 dilution, Santa Cruz Biotechnology) and a peroxidase-labeled anti-mouse immunoglobulin (Ig) G antibody (1:1000 dilution, Santa Cruz Biotechnology, CA, USA). The relative density of membrane to cytosolic RhoA was determined using NIH imaging software.

Expression of 5-HTT and RhoA/ROCK and Phosphorylation of ERK1/2 and AKT Kinase

The expression of RhoA/ROCK and phosphorylation of ERK1/2 and AKT kinase in PASMCs were assessed by incubating the PASMCs with 5-HT (10 μM) for 15 min (peak time for RhoA/ROCK, ERK1/2 and AKT phosphorylation) and 5-HTT for 10 min (peak time for 5-HTT) after KMUP-1 (1-100 μM) was added to the culture of PASMCs for 24 h.

Expression of eNOS and 5-HT_{2B} Receptor in HPAEC

To measure the expression of eNOS and 5-HT_{2B} receptor in HPAEC after incubation with KMUP-1 (1-100 μM) for 24 h, whole lysates were collected for Western blotting assay. The relative density was determined using NIH imaging software.

Nitric Oxide Production in HPAEC

Production of NO in HPAEC was determined using the Griess method. The three step Greiss test converts nitrate (NO₃⁻) into nitrite (NO₂⁻) giving a total NO₂⁻ concentration from a standard calibration curve. HPAEC were incubated with or without KMUP-1 (10 μM) for 24 h and then incubated with 5-HT (10 μmol/L) for 30 min. Media samples from HPAEC were taken and the NO concentration was determined. Six independent experiments were carried out and data are reported as the mean mean±SEM.

Cell Migration Assay Under Microscope

Migration of PASMCs was assessed using a wound assay model in which cells grown to confluence on 6 well dishes were scraped with the edge of a fine razor. The wound edge was viewed and photographed under a microscope (Nikon, Tokyo, Japan) before and after culture for 24 h in serum-free DMEM in the presence of 5HT (10 μM). The distance the cells migrated from the wound surface was then manually measured.

Displacement of Radioligand Binding Assay

As described previously, full length clones of 5-HT_{2A}, 5-HT_{2B} and 5-HT_{2C} receptor were prepared from CHO-K1 cells. On the basis of higher affinity with [³H]-radiolabeled ketanserin, [³H]-radiolabeled lysergic acid diethylamide (LSD) and [³H]-radiolabeled mesulergine (0.15-1.2 nM) measured the gene products of human 5-HT_{2A} and 5-HT_{2B} receptor and 5-HT_{2C} receptor; respectively, were used to test KMUP-1's effects on radioligand binding ability. IC₅₀ represents the concentration of competing ligand which displaces 50% of the specific binding of the radioligand. Kᵢ values for competition curves were calculated using the Cheng-Prusoff equation (Cheng, 2001).

Short Term Nebulization

Short-term KMUP-1 (5 mM, 30 min) nebulization and pretreatment with L-NAME (12 mM, 30 min) were accomplished using a previously described method (Alp S, et al., 2006). Aerosols were generated by ultrasonic nebulizer from day 21 to 27. Mice were sacrificed on day 28, (Cataldo D D, et al., 2002). Three different experiments were performed on cohorts of 7˜14 mice per experimental condition.

Allergen-Sensitization, Challenge and Treatment

In this long-term experiment, 6-8 week old BALB/c female mice were sensitized on days 1 and 8 by intra-peritoneal injection (i.p.) of ovalbumine (10 μg) (Sigma-Aldrich, Schnelldorf, Germany) adsorbed to 2 mg aluminum hydroxide (AlumInject; Perbio, Erembodegem, Belgium). Mice were divided into three groups. In Group 1, a control group was only exposed to saline nebulization by aerosol. The other 2 groups were subjected to ovalbumine (1%) nebulization. Before each challenge with ovalbumine, mice were either exposed to saline nebulization (Group 2) or KMUP-1 nebulization (5 mM) for 30 min by aerosol (Group 3).

Bronchoalveolar Lavage and Cell Counts

After the sacrifice of mice, a cannula was placed in the trachea for bronchoalveolar lavage by gentle manual aspiration, using cold PBS-EDTA (0.05 mM, 1 ml) (Calbiochem, Darmstadt, Germany) as previously described (Cataldo D D, et al., 2002). The broncho alveolar lavage fluid (BALF) was centrifuged at 4° C. for 10 min (1200 rpm). The supernatant was frozen at −80° C. for protein assessment and the cell pellet was resuspended in PBS-EDTA (0.05 mM, 1 ml). Total cell counts were performed manually using a thoma chamber. The differential cell counts were performed using morphologic criteria on cytocentrifuged preparations (Cytospin) after staining with Diff-Quick (Dade, Belgium). Differential cell counts were performed by one blinded observer unaware of the experimental treatment.

Zymography

The gelatinolytic activity of MMP-9 was measured by gelatin zymography as described previously (Cataldo D D, et al., 2002). Briefly, BALF (10 μl) was subjected to electrophoresis in 10% polyacrylamide gel containing gelatin (1 mg/ml). The gel was washed in 2.5% Triton X-100 to permit re-naturation of gelatinases and stained with Coomassie blue after overnight incubation. Destaining visualized clear zones of lysis against a blue background indicated the gelatinase activity (Hibbs, M S, et al., 1985).

Western Blotting Analysis

The lung tissues were homogenized, washed with PBS and incubated in lysis buffer in addition to a protease inhibitor cocktail (Sigma, St. Louis, Mo.) to obtain extracts of lung proteins. A Western blot analysis was performed as described previously (Wu B N, et al., 2006). The samples were loaded to 10% SDS-PAGE gels and were separated at 120 V for 90 min. The blots were incubated with anti-eNOS antibody diluted at a ratio of 1:500, anti-sGCα1 antibody diluted at a ratio of 1:2000, anti-PKG antibody diluted at a ratio of 1:500, anti-VCAM-1 antibody diluted at a ratio of 1:500, anti-ICAM-1 antibody diluted at a ratio of 1:500, and anti-MMP-9 antibody diluted at a ratio of 1:600 overnight at 4° C. The membranes were stripped and re-blotted with anti-actin antibody (Sigma, St. Louis, Mo.) to verify the equal loading of protein in each lane.

NO metabolites (NOx)

The remaining BALF was centrifuged (900 g, 1200 rpm, 10 min) and the supernatant frozen (−80° C.) until determination of nitrite and nitrate by the Griess reaction. BALF (100 μL) was incubated (37° C.) for 30 min with N-2-hydroxyethylpiperazine-N′-ethane sulphonic acid buffer (HEPES) (50 mM, pH 7.4), flavin adenine dinucleotide (5 μM), nicotinamide adenine dinucleotide phosphate (0.1 mM), distilled water (290 μl), and nitrate reductase (0.2 U/ml) for the conversion of nitrate into nitrite. The nitrate reductase was absent for nitrite determination. Unreacted nicotinamide adenine dinucleotide phosphate was oxidized by incubating (25° C., 10 min) with potassium ferricyanide (1 mM). The samples were then incubated (25° C., 10 min) with Griess reagent [N-(1-naphthyl)-ethylenediamine: 0.2% (w/v), sulphanilamide: 2% (w/v), solubilized in double-distilled water: 95% and phosphoric acid: 5% (v/v)] and the absorbance measured at 540 nm. The levels of nitrite in the BALF were calibrated and compared to a sodium nitrite (0˜150 μl) standard curve.

Histologic and Morphologic Analyses

Sections of fixed embedded tissues were cut at 4 mm, placed on glass slides, deparaffinized and stained sequentially with hematoxylin 2 and eosin-Y (MUTO PURE CHEMICALS CO., LTD., Tokyo, Japan) (Tournoy K G, et al., 2000). To confirm the relationship between vascular inflammation/remodeling and expression of proteins, lung tissue sections from ovalbumine experiments were prepared and stained with hematoxylin-eosin as previously described (Hongo M, et al., 2000). Microphotography analysis of blood vessels and bronchioles, focusing on peri-bronchial and peri-vascular region in the lung sections, was performed using a color digital camera mounted on a computer-interfaced light microscope (Eclipse TE2000-S microscope, Nikon, Tokyo, Japan).

Vascular and Bronchial Wall Thickening

The thickness of vascular and bronchial walls was measured as the distance between the external and internal elastic laminae of each artery or bronchiole, and the external diameter was measured as the diameter of the external lamina using a calibrated eyepiece micrometer. The percent wall thickness (%) of each artery or bronchiole was calculated using the following formula: Wall thickness (%)=(2×medial wall thickness)/(external diameter)×100. The average of six measurements of pulmonary artery or bronchiole in each animal was used in analysis.

eNOS- and MMP-9-Immunohistochemical Staining

Immunohistochemical analysis of eNOS enhancement by KMUP-1 was performed as follows. The lungs were perfused with saline followed by 10% formalin and then placed in 10% formalin for paraffin embedding. The lung tissues were cut into 3˜5 mm sections onto slides, dewaxed in 100% xylene, and then re-hydrated in graded alcohol solutions. Throughout the procedures, slides were washed as appropriate in PBS. Antigen retrieval was performed by microwave treatment for eNOS immunostaining. Sections were treated with 3% H₂O₂ (10 min) to inhibit endogenous peroxidases and incubated with normal serum (60 min) to reduce nonspecific binding of secondary antibodies. Sections were then incubated at 4° C. overnight with eNOS antibodies. After washing off unbounded primary antibodies, sections were incubated with secondary biotinylated antibodies against mouse or rabbit (DAKO Co., Japan) (60 min), followed by incubation in avidin/biotin/horseradish peroxidase complex (DAKO Co., Japan) (30 min) Subsequently, peroxidase activity was visualized by incubation (3 min) with 0.05% 3,3′-diaminobenzidine (DAKO Co., Japan), which gives a brown reaction product. The reaction was stopped by washing with water. The slides were counterstained with hematoxylin. For immunostaining of MMP-9 in lung tissue, the de-paraffinized 3-5 μm sections of lung tissue was performed as above eNOS, but using MMP-9 antibody.

Measurement of Cytokines

To confirm whether allergen-sensitized and challenged mice is involved in the response to immunologic activation, we measure the plasma cytokines IL-5 and IL-13 of mice and treatment with KMUP-1 by ELISAS techniques, according to the manufacturer's guidance.

### Example 1

### Preparation of KMUP-1 HCl salt (7-[2-[4-(2-chlorophenyl)piperazinyl]ethyl]-1,3-dimethylxanthine HCl)

KMUP-1 (8.0 g) is dissolved in a mixture of ethanol (10 mL) and 1N HCl (60 mL) for reacting at 50° C. for 10 min. The methanol is added into the solution under room temperature and the solution is incubated over night for crystallization. The crystal is filtrated to obtain the precipitate of KMUP-1 HCl salt (7.4 g).

### Example 2

### Preparation of KMUP-3 HCl salt (7-[2-[4-(4-nitrobenzene)piperazinyl]ethyl]-1,3-dimethyl xanthine HCl)

KMUP-3 (8.3 g) is dissolved in a mixture of ethanol (10 mL) and 1N HCl (60 mL) The solution is reacted at 50° C. for 20 min, the methanol is added thereinto under room temperature, and the solution is incubated over night for crystallization and filtrated to obtain KMUP-3 HCl salt (6.4 g).

### Example 3

### Preparation of KMUP-1-γ-Polyglutamic acid salt

Method 1:

Sodium γ-polyglutamic acid (20 g) is suspended in distill water and added with KMUP-1 HCl (16 g) dissolved in methanol 100 ml to reflux in a three-neck reactor, equipped with a condenser, for 1 hour. After cooling, obtained precipitate is dissolved in methanol 100 ml and the resulted solution is incubated for crystallization and filtrated to obtain KMUP-1-γ-Polyglutamic acid salt (35.6 g).

Method 2:

KMUP-1 HCl (16 g) is dissolved in methanol 100 ml and added with sodium alginic acid 20 g dissolved in methanol 100 ml to reflux in a three-neck reactor, equipped with a condenser, for 1 hour. After cooling, obtained precipitate is filtrated and re-crystallized with methanol 100 ml to have KMUP-1-γ-Polyglutamic acid salt (35.8 g).

### Example 4

### Preparation of KMUP-3-Nicotinic acid salt

KMUP-3 (8.3 g) is dissolved in a mixture of ethanol (10 mL) and Nicotinic acid (2.4 g). The solution is reacted at 50° C. for 20 min, the methanol is added thereinto under room temperature, and the solution is incubated over night for crystallization and filtrated to obtain KMUP-3-Nicotinic acid salt (8.3 g).

### Example 5

### Preparation of KMUP-1-Simvastatinic Complex

KMUP-1 (8.0 g) is dissolved in a mixture of ethanol (10 mL) and HCl (1N, 60 mL) and reacted at 50° C. for 10 min, the methanol is added thereinto under room temperature, and the solution is incubated over night for crystallization and filtrated to obtain KMUP-1 HCl (7.4 g). Take KMUP-1 HCl salt (4.4 g) and redissolve it in ethanol (150 mL) for use.

In a flask equipped with a magnetic stirrer, simvastatin (4.2 g) dissolved in ethanol (50 ml) is poured, to which an aqueous solution of sodium hydroxide (4 g/60 ml) and the above-mentioned filtrate of KMUP-1 HCl salt reacted with the ethanol are added under room temperature. The mixture is reacted at 50° C. for 20 mins, rapidly filtrated and incubated one hour for crystallization to give the KMUP-1-Simastatinic complex.

### Example 6

### Preparation of KMUP-2-polyacrylic acid salt

KMUP-2 HCl (8 g) is dissolved in a mixture of methanol (100 mL) and sodium polyacrylic acid (2.5 g). The solution is refluxed in a three-neck reactor, equipped with a condenser, for 1 hour. After cooling, obtained precipitate is re-dissolved in methanol 100 ml, and the resulted solution is incubated for crystallization and filtrated to obtain KMUP-2-polyacrylic acid salt (7.4 g).

### Example 7

### Preparation of the Composition in Tablet

Tablets are prepared using standard mixing and formation techniques as described in U.S. Pat. No. 5,358,941, to Bechard et al., issued Oct. 25, 1994, which is incorporated by reference herein in its entirety.
KMUP-3 Citric acid salt 100 mg
Lactose qs
Corn starch qs

### Example 8

### Preparation of the Composition in Tablet

Tablets are prepared using standard mixing and formation techniques as described in U.S. Pat. No. 5,358,941, to Bechard et al., issued Oct. 25, 1994, which is incorporated by reference herein in its entirety.
Simvastatin 50 mg
Lactose qs
Corn starch qs

### Example 9

### Preparation of the Aerosolized Formulation

Aerosols are prepared using standard mixing and formation techniques as described in U.S. Pat. No. 6,713,047, to David et al., issued Mar. 30, 2004, which is incorporated by reference herein in its entirety.
KMUP-1 HCl 5 mg/shot
Glycerol 1.3% (w/w)
HFA to 12 ml

### Embodiments

1. An obstructive pulmonary disease inhibiting pharmaceutical composition including:
an effective amount of a compound of formula I, wherein
R₂ and R₄ are each selected independently from the group consisting of a C1˜C5 alkoxy group, a hydrogen, a nitro group, and a halogen atom; and
a pharmaceutically acceptable carrier.

2. The pharmaceutical composition of Embodiment 1, wherein the halogen atom is one selected from a group consisting of a fluorine, a chlorine, a bromine and an iodine.

3. The pharmaceutical composition of any one of Embodiments 1-2, wherein the compound of formula I is KMUPS derivative compound.

4. The pharmaceutical composition of any one of Embodiments 1-3, wherein the KMUPS derivative compound is one selected from a group consisting of KMUP-1, KMUP-2, KMUP-3, KMUP-4 and its pharmaceutical acceptable salts.

5. A method of providing a medical effect for inhibiting of pulmonary inflammatory, comprising steps of:
providing a subject in need thereof; and
administering an effective amount of pharmaceutical composition of KMUPS derivative to the subject in need thereof.

6. A method as Embodiments 5, wherein the KMUPS derivative compounds is one selected from a group consisting of KMUP-1, KMUP-2, KMUP-3 and KMUP-4.

7. A method as Embodiments 5, wherein the administration is performed by one selected from an oral, injection, inhalation and topical administration.

8. A method for inhibiting of pulmonary inflammatory as Embodiments 5, comprising a step of:
combination administrating a pharmaceutically effective amount of a compound of KMUPS derivative and a statins analogues to a mammal in need thereof.

9. A method as Embodiments 5, wherein the statins analogues is one selected from a group consisting of Atorvastatin, Cerivastatin, Fluvastatin, Lovastatin, Mevastatin, Pravastatin, Rosuvastatin, Simvastatin and Pramastatinic acid.

10. An obstructive pulmonary disease inhibiting pharmaceutical composition including:
an effective amount of a compound of 7-[2-[4-(2-chlorobenzene)piperazinyl]ethyl]-1,3-dimethylxanthine-Citric acid; and a pharmaceutically acceptable carrier.

11. An obstructive pulmonary disease inhibiting pharmaceutical composition including:
an effective amount of a compound of 7-[2-[4-(2-chlorobenzene)piperazinyl]ethyl]-1,3-dimethylxanthine-Nicotinic acid; and
a pharmaceutically acceptable carrier.

12. An obstructive pulmonary disease inhibiting pharmaceutical composition including:
an effective amount of a compound of 7-[2-[4-(2-methoxybenzene)piperazinyl]ethyl]-1,3-dimethylxanthine hydrochloride; and
a pharmaceutically acceptable carrier.

13. An obstructive pulmonary disease inhibiting pharmaceutical composition including:
an effective amount of a compound of 7-[2-[4-(2-methoxybenzene)piperazinyl]ethyl]-1,3-dimethylxanthine-Citric acid;
and
a pharmaceutically acceptable carrier.

14. An obstructive pulmonary disease inhibiting pharmaceutical composition including:
an effective amount of a compound of 7-[2-[4-(2-methoxybenzene)piperazinyl]ethyl]-1,3-dimethylxanthine-Nicotinic acid; and
a pharmaceutically acceptable carrier.

15. An obstructive pulmonary disease inhibiting pharmaceutical composition including:
an effective amount of a compound of 7-[2-[4-(4-nitrobenzene)piperazinyl]ethyl]-1,3-dimethylxanthine hydrochloride;
and
a pharmaceutically acceptable carrier.

16. An obstructive pulmonary disease inhibiting pharmaceutical composition including:
an effective amount of a compound of 7-[2-[4-(4-nitrobenzene)piperazinyl]ethyl]-1,3-dimethylxanthine-Citric acid; and
a pharmaceutically acceptable carrier.

17. An obstructive pulmonary disease inhibiting pharmaceutical composition including:
an effective amount of a compound of 7-[2-[4-(4-nitrobenzene)piperazinyl]ethyl]-1,3-dimethylxanthine-Nicotinic acid;
and
a pharmaceutically acceptable carrier.

18. An obstructive pulmonary disease inhibiting pharmaceutical composition including:
an effective amount of a compound of 7-[2-[4-(2-nitrobenzene)piperazinyl]ethyl]-1,3-dimethylxanthine hydrochloride; and
a pharmaceutically acceptable carrier.

19. An obstructive pulmonary disease inhibiting pharmaceutical composition including:
an effective amount of a compound of 7-[2-[4-(2-nitrobenzene)piperazinyl]ethyl]-1,3-dimethylxanthine-Citric acid;
and
a pharmaceutically acceptable carrier.

20. An obstructive pulmonary disease inhibiting pharmaceutical composition including:
an effective amount of a compound of 7-[2-[4-(2-nitrobenzene)piperazinyl]ethyl]-1,3-dimethylxanthine-Nicotinic acid; and
a pharmaceutically acceptable carrier.

21. An anti-pulmonary artery hypertension pharmaceutical composition including:

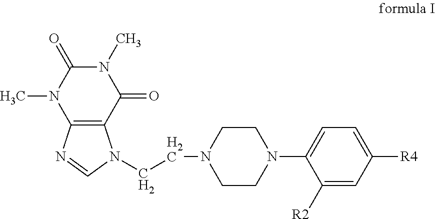

an effective amount of a compound of formula I, wherein
R₂ and R₄ are each selected independently from the group consisting of a C1˜C5 alkoxy group, a hydrogen, a nitro group, and a halogen atom; and
a pharmaceutically acceptable carrier.

22. The pharmaceutical composition of Embodiment 21, wherein the halogen atom is one selected from a group consisting of a fluorine, a chlorine, a bromine and an iodine.

23. The pharmaceutical composition of any one of Embodiments 21-22, wherein the compound of formula I is KMUPS derivative compound.

24. The pharmaceutical composition of any one of Embodiments 21-23, wherein the KMUPS derivative compound is one selected from a group consisting of KMUP-1, KMUP-2, KMUP-3, KMUP-4 and its pharmaceutical acceptable salts.

25. The pharmaceutical composition of Embodiments 21-24, wherein the compound is for treating acute or chronic pulmonary artery hypertension.

26. A combination therapy method of providing a medical effect for inhibiting of pulmonary inflammatory, comprising steps of:
providing a subject in need thereof; and
administering an effective amount of pharmaceutical composition of a KMUPS derivative and a statins analogues to the subject in need thereof.

27. The method of Embodiment 26, the pharmaceutical composition comprising:

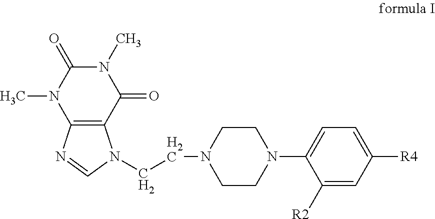

an effective amount of a KMUPS derivative compound of formula I, wherein R₂ and R₄ are each selected independently from the group consisting of a C1˜C5 alkoxy group, a hydrogen; a nitro group, and a halogen atom; and
a pharmaceutically acceptable carrier.

28. The method of any one of Embodiments 26-27, wherein the halogen atom is one selected from a group consisting of a fluorine, a chlorine, a bromine and an iodine.

29. The method of any one of Embodiments 26-28, wherein the pharmaceutical composition of KMUPS derivative compounds is one selected from a group consisting of KMUP-1, KMUP-2, KMUP-3, KMUP-4 and its pharmaceutical acceptable salts.

30. The method of any one of Embodiments 26-29, wherein the pharmaceutical composition of statins analogues is one selected from a group consisting of Atorvastatin, Cerivastatin, Fluvastatin, Lovastatin, Mevastatin, Pravastatin, Rosuvastatin, Simvastatin, Pramastatinic acid and its pharmaceutical acceptable salts.

31. A combination therapy method of providing a medical effect for preventing of allergic pulmonary vascular inflammation, comprising steps of:
providing a subject in need thereof; and
administering an effective amount of pharmaceutical composition of a KMUPS derivative and a statins analogues to the subject in need thereof.

32. The method of Embodiment 31. the pharmaceutical composition comprising:

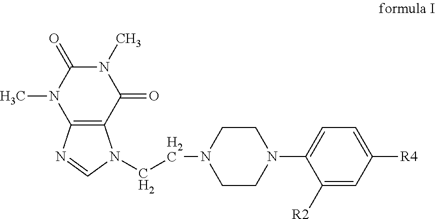

an effective amount of a KMUPS derivative compound of formula I, wherein
R₂ and R₄ are each selected independently from the group consisting of a C1˜C5 alkoxy group, a hydrogen; a nitro group, and a halogen atom; and
a pharmaceutically acceptable carrier.

33. The method of any one of Embodiments 31-32, wherein the halogen atom is one selected from a group consisting of a fluorine, a chlorine, a bromine and an iodine.

34. The method of any one of Embodiments 31-33, the pharmaceutical composition of KMUPS derivative compounds is one selected from a group consisting of KMUP-1, KMUP-2, KMUP-3, KMUP-4 and its pharmaceutical acceptable salts.

35. The method of any one of Embodiments 31-34, the pharmaceutical composition of statins analogues is one selected from a group consisting of Atorvastatin, Cerivastatin, Fluvastatin, Lovastatin, Mevastatin, Pravastatin, Rosuvastatin, Simvastatin, Pramastatinic acid and its pharmaceutical acceptable salts.

36. The method of any one of Embodiments 31-35, wherein the pharmaceutical composition of a KMUPS derivative and a statins analogues are formulated independently.

37. The method of any one of Embodiments 31-36, wherein the pharmaceutical composition of a KMUPS derivative and a statins analogues are formulated independently.

38. A method for inhibiting a physiological activity of a lung cell, comprising a step of:
combination administrating a pharmaceutically effective amount of a compound of KMUPS derivative and a statins analogues to a mammal in need,
   wherein the compound is a medical effect for preventing of allergic pulmonary vascular remodeling and Via NO, Suppressed MMP-9 And ICAM-1/VCAM-1 and a combination thereof.

39. The method of Embodiment 38, the pharmaceutical composition comprising:
an effective amount of KMUPS complex compound represented by one of formula II and formula III, wherein R₂ and R₄ are each selected independently from the group consisting of a C1˜C5 alkoxy group, a hydrogen; a nitro group, and a halogen atom; and
a pharmaceutically acceptable carrier.

40. The method of any one of Embodiments 38-39, wherein the halogen atom is one selected from a group consisting of a fluorine, a chlorine, a bromine and an iodine.

41. The method of any one of Embodiments 38-40, wherein the pharmaceutical composition of KMUPS derivative compounds is one selected from a group consisting of KMUP-1, KMUP-2, KMUP-3, KMUP-4 and its pharmaceutical acceptable salts.

42. The method of any one of Embodiments 38-41, wherein the pharmaceutical composition of statins analogues is one selected from a group consisting of Atorvastatin, Cerivastatin, Fluvastatin, Lovastatin, Mevastatin, Pravastatin, Rosuvastatin, Simvastatin, Pramastatinic acid and its pharmaceutical acceptable salts.

43. The method of any one of Embodiments 38-42, wherein the pharmaceutical composition of a KMUPS derivative and a statins analogues are formulated independently.

### REFERENCES

1. Alp S, Skrygan M, Schmidt W E, Bastian A, Pulm Pharmacol Ther 2006; 19:386-390.
2. Cataldo D D, Tournoy K G, Vermaelen K, Munaut C, Foidart J M, Louis R, et al. Am J Pathol 2002; 61:491-498.
3. Hibbs, M. S., Hasty, K. A., Seyer, J. M., Kang, A. H. and Mainardi, C. L. J Biol Chem 1985; 260: 2493-2500.
4. Hongo M, Ryoke T, Schoenfeld J, Hunter J, Dalton N, Clark R, Lowe D, Chien K, Ross J. Jr. Basic Res Cardiol 2000; 95:431-441.
5. Lin R J, Wu B N, Lo Y C, An L M, Dai Z K, Lin Y T, Tang C S, Chen I J. J Pharmacol Exp Ther 2006; 316:709-717.
6. Liu C P, et al., 2012, Inflammation & allergy drug target 2012; In press.
7. Tournoy K G, Kips J C, Schou C, Pauwels R A. Clin Exp Allergy 2000; 30:79-85.
8. Toward T J, Smith N, Broadley K J. Am. J Respir Crit. Care Med. 2004; 169: 227-234.
9. Wu B N, Chen C W, Liou S F, Yeh J L, Chung H H, Chen I J, Mol Pharmacol 2006; 70:977-985.
10. Wu B N, Chen H Y, Liu C P, Hsu, L Y, Chen, I J, International Journal of immunopathol and Pharmacol 2011; 24:925-940.

What is claimed is:

1. A method for inhibiting a pulmonary inflammation, comprising steps of:
providing a subject in need thereof; and
administering one selected from a group consisting of KMUPS compound represented by formula I, a pharmaceutically acceptable salts thereof;
and a pharmaceutical composition thereof to the subject in a dosage from 1 to 2.5 milligram per kilogram of body weight, 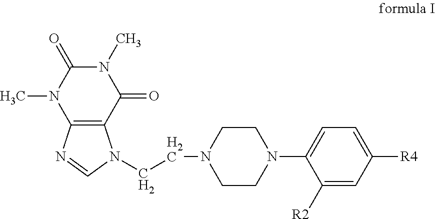
wherein R₂ and R₄ are each selected independently from a group consisting of a C1˜C5 alkoxy group, a hydrogen, a nitro group and a halogen atom.

2. A method as claimed in claim 1, wherein the KMUPS compound comprising is one selected from a group consisting of KMUP-1, KMUP-2, KMUP-3 and KMUP-4.

3. A method as claimed in claim 2, wherein KMUP-1 is 7-[2-[4-(2-chlorobenzene) piperazinyl]ethyl]-1,3-dimethylxanthine, KMUP-2 is 7-[2-[4-(2-methoxybenzene)piperazinyl]ethyl]-1,3-dimethylxanthine, KMUP-3 is 7-[2-[4-(4-nitrobenzene)piperazinyl]ethyl]-1,3-dimethylxanthine and KMUP-4 is 7-[2-[4-(2-nitrobenzene)piperazinyl]ethyl]-1,3-dimethylxanthine.

4. A method as claimed in claim 1, wherein the pulmonary inflammation comprising one selected from a group consisting of an obstructive pulmonary disease, a pulmonary artery hypertension, pulmonary artery proliferation, an allergic pulmonary vascular inflammation, a physiological activity of a lung cell, a vascular remodeling inhibiting disease and a combination thereof.

5. A method as claimed in claim 1, wherein the administering steps is performed by an oral administration.

6. A method for inhibiting a pulmonary inflammation, comprising steps of:
providing a subject in need thereof; and
administering one of a KMUPS complex compound represented by one of formula II and formula III, and a pharmaceutical composition thereof to the subject in need thereof in a dosage from 1 to 2.5 milligram per kilogram of body weight, 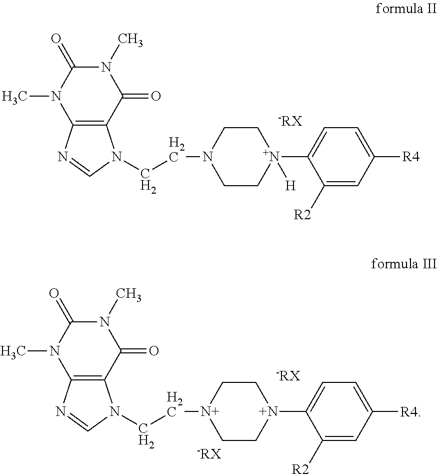
wherein: R2 and R4 are each selected independently from a group consisting of a C1˜C5 alkoxy group, a hydrogen, a nitro group, and a halogen atom;
RX includes a carboxylic group selected from a group comprising of a Statin analogues, a Co-polymer, a poly-γ-polyglutamic acid derivative and a sodium CMC; and
⁻RX is an anion of a carboxylic group donated from one selected from a group comprising of a Statin analogues, a Co-polymer, a poly-γ-polyglutamic acid derivative and a sodium CMC.

7. A method as claimed in claim 6, wherein the KMUPS complex compound is one selected from a group consisting of KMUP-1-Atorvastatin complex, KMUP-2-Atorvastatin complex, KMUP-3-Atorvastatin complex, KMUP-4-Atorvastatin complex, KMUP-1-Cerivastatin complex, KMUP-2-Cerivastatin complex, KMUP-3-Cerivastatin complex, KMUP-4-Cerivastatin complex; KMUP-1-Fluvastatin complex, KMUP-2-Fluvastatin complex, KMUP-3-Fluvastatin complex, KMUP-4-Fluvastatin complex, KMUP-1-Lovastatin complex, KMUP-2-Lovastatin complex, KMUP-3-Lovastatin complex, KMUP-4-Lovastatin complex; KMUP-1-Mevastatin complex, KMUP-2-Mevastatin complex, KMUP-3-Mevastatin complex, KMUP-4-Mevastatin complex, KMUP-1-Pravastatin complex, KMUP-2-Pravastatin complex, KMUP-3-Pravastatin complex, KMUP-4-Pravastatin complex, KMUP-1-Rosuvastatin complex, KMUP-2-Rosuvastatin complex, KMUP-3-Rosuvastatin complex, KMUP-4-Rosuvastatin complex, KMUP-1-Simvastatin complex, KMUP-2-Simvastatin complex, KMUP-3-Simvastatin complex, KMUP-4-Simvastatin complex, KMUP-1-CMC complex, KMUP-2-CMC complex, KMUP-3-CMC complex, KMUP-4-CMC complex, KMUP-1-hyaluronic complex, KMUP-2-hyaluronic complex, KMUP-3-hyaluronic complex, KMUP-4-hyaluronic complex, KMUP-1-polyacrylic complex, KMUP-2-polyacrylic complex, KMUP-3-polyacrylic complex, KMUP-4-polyacrylic complex, KMUP-1-Eudragit complex, KMUP-2-Eudragit complex, KMUP-3-Eudragit complex, KMUP-4-Eudragit complex; KMUP-1-polylactide complex, KMUP-2-polylactide complex, KMUP-3-polylactide complex, KMUP-4-polylactide complex; KMUP-1-polyglycolic complex, KMUP-2-polyglycolic complex, KMUP-3-polyglycolic complex, KMUP-4-polyglycolic complex, KMUP-1-dextran sulfate complex, KMUP-2-dextran sulfate complex, KMUP-3-dextran sulfate complex, KMUP-4-dextran sulfate complex, KMUP-1-heparan sulfate complex, KMUP-2-heparan sulfate complex, KMUP-3-heparan sulfate complex, KMUP-4-heparan sulfate complex, KMUP-1-alginate complex, KMUP-2-alginate complex, KMUP-3-alginate complex, KMUP-4-alginate complex, KMUP-1-γ-PGA complex, KMUP-2-γ-PGA complex, KMUP-3-γ-PGA complex, KMUP-4-γ-PGA complex, KMUP-1-APA complex, KMUP-2-APA complex, KMUP-3-APA complex and KMUP-4-APA complex.

8. A method as claimed in claim 6, wherein the pulmonary inflammation comprises one selected from a group consisting of an obstructive pulmonary disease, a pulmonary artery hypertension, pulmonary artery proliferation, an allergic pulmonary vascular inflammation, a physiological activity of a lung cell, a vascular remodeling inhibiting disease and a combination thereof.
